(19) United States
(12) Patent Application Publication
(10) Pub. No.: US 2012/0202744 A1
(43) Pub. Date: Aug. 9, 2012

(54) DUAL ANTAGONISM OF ENDOTHELIN TYPE A AND BRADYKININ B1 RECEPTORS FOR TREATING PAIN AND PREVENTING CARTILAGE DEGRADATION

(76) Inventors: Florina MOLDOVAN, Montreal (CA); Gabriel Nathan Kaufman, Montreal (CA); Pierre Sirois, Orford (CA)

(21) Appl. No.: 13/366,036

(22) Filed: Feb. 3, 2012

Related U.S. Application Data
(60) Provisional application No. 61/439,517, filed on Feb. 4, 2011.

(51) Int. Cl. A61K 38/12; A61P 29/00; A61P 19/02; A61P 25/04

(52) U.S. Cl. 514/16.8; 514/21.1; 514/18.3

(57) ABSTRACT

The present invention relates to the field of anatomy, and more particularly to the control of pain and to the prevention and treatment of osteoarthritic cartilage degradation. Described herein are methods for treating pain which comprise administering to a subject in need a combination of (i) an antagonist of the endothelin type A receptor (ETA) and (ii) an antagonist of the bradykinin B1 receptor (BKB1). Also described are methods for preventing osteoarthritic cartilage degradation and pharmaceutical compositions for treating pain, for preventing osteoarthritic cartilage degradation, and/or for preventing osteoarthritic joint inflammation, in subjects.

### FIELD OF INVENTION

The present invention relates to the field of anatomy, and more particularly to the control of pain and to the prevention and treatment of osteoarthritic cartilage degradation.

### BACKGROUND OF INVENTION

Osteoarthritis (OA) is a pathology, which causes severe and long-term pain and physical disability, which is without an effective treatment. This disorder is classified as a non-inflammatory arthritis and it is the most frequent joint disorder in seniors. Osteoarthritis is the most common form of arthritis, and can affect people of all ages, including children. Most often involving the hands and the large weight-bearing joints (hips, knees, back), OA is clinically characterized by the progressive destruction of articular cartilage, subchondral bone remodelling, osteophyte formation, and synovial membrane changes. The pathophysiological mechanisms responsible for these changes are not yet completely understood. This leads to joint pain, which can lead to reduced physical activity and thereby raise the risk of other diseases. The medical treatment options for OA are mainly limited to symptom alleviation, with no disease-modifying drugs currently available for clinical use. In severe cases, joint replacement surgery is necessary, which represents a significant health-care burden.

Previous studies have demonstrated that cartilage degradation (catabolism) and chondrocyte metabolism are affected in OA. In advanced OA, this results in the complete loss of articular cartilage and in the formation of large lesions with bone exposure. Endothelin-1, the vasoconstrictor peptide, influences cartilage metabolism via endothelin receptor type A (ETA) (Khatib A M, Lomri A, Mitrovic R D, Moldovan F: Articular chondrocyte aging and endothelin-1. Cytokine 2007, 37:6-13 and Messai H, Panasyuk A, Khatib A, Barbara A, Mitrovic DR: Endothelin-1 receptors on cultured rat articular chondrocytes: regulation by age, growth factors, and cytokines, and effect on cAMP production. Mech Ageing Dev 2001, 122(6):519-31.). In addition, it has been suggested that the bradykinin system, through expression of the bradykinin B1 receptor (BKB1), plays a role in local tissue injury, inflammation, and nociception. Sainz et al. have shown that, in an animal model of induced inflammatory arthritis, bradykinin influences chronic inflammation through ligation of both BKB1 and bradykinin b2 (BKB2) receptors (Sainz I M, Uknis A B, Isordia-Salas I, Dela Cadena R A, Pixley R A, Colman R W. Interactions between bradykinin (BK) and cell adhesion molecule (CAM) expression in peptidoglycan-polysaccharide (PG-PS)-induced arthritis. FASEB J. 2004 May; 18(7):887-9.). Numerous antagonists of the Endothelin type A receptor are known and have been described in Aubert et. al., Expert Opin. Ther. Targets (2009), 13(9): 1069-1084. Numerous antagonists of the bradykinin b1 receptor are known also and have been described in Fincham et al., Expert Opin. Ther. Patents (2009), 19(7): 919-941.

In previous poster presentations, the inventors disclosed results where ETA and BKB1 peptide antagonists were studied in both in vitro and in vivo osteoarthritis models (Selective endothelin receptor A and bradykinin receptor 1 antagonists in osteoarthritis treatment, Kaufman G N, Zaouter C, Londono I, Moldovan F. Poster presented at: 1) OsteoArthritis Research Society International 2009 World Congress, Sep. 10-13, 2009 Montreal, Quebec; and 2) APS 11th International Conference on Endothelin, Sep. 9-12, 2009 Montreal, Quebec). However, these previous studies do not address a potential role for ETA and BKB1 peptide antagonists in alleviating joint pain, nor in preventing osteoarthritic cartilage degradation. It is only recently that the inventors published a manuscript showing that BKB1 antagonism improves nociceptive tolerance, and both ETA and/or BKB1 antagonism prevents joint cartilage degradation in a surgical model of osteoarthritis (Kaufman et al., Arthritis Research and Therapy, 2011, 13:R76).

It is known that BKB1 antagonists can be used to treat various conditions where BKB1 receptors are expressed. For instance, international patent publication WO 2006/017938 and U.S. Pat. No. 7,211,566 describe BKB1 receptor antagonist compounds that may be used for the treatment of inflammation due to diabetic vasculopathy (which is related to microvascular leakage) and inflammatory lung cell infiltration and activation in allergic asthma. However, these patent documents do not suggest using the compounds described therein for alleviating joint pain, nor for preventing osteoarthritic cartilage degradation. Nor do they suggest using the compounds described therein in conjunction with endothelin receptor antagonists.

Therefore, there is a need for relieving pain in patients, more particularly in patients afflicted with osteoarthritis. There is also a need for pharmaceutical compositions for protecting cartilage tissue from injury, for decreasing joint pain and for abrogating joint inflammation.

The present invention addresses these needs, as it provides methods, compounds, compositions, and treatment approaches for alleviating joint pain and for preventing osteoarthritic cartilage degradation.

Additional features of the invention will be apparent from review of the disclosure, figures, and description of the invention below.

### BRIEF SUMMARY OF THE INVENTION

One aspect of the present invention relates to methods, compounds and composition for treating pain in a subject in need thereof. In one embodiment, the method for treating pain comprises administering to the subject a combination of (i) an antagonist of the endothelin type A receptor (ETA) and (ii) an antagonist of the bradykinin B1 receptor (BKB1). In a particular embodiment the pain is joint pain. In another embodiment, the subject is suffering from osteoarthritis.

Another aspect of the present invention relates to methods, compounds and composition for preventing and/or treating osteoarthritic cartilage degradation in a subject in need thereof. In one embodiment, the method for preventing osteoarthritic cartilage degradation comprises administering to the subject a combination of (i) an antagonist of the endothelin type A receptor (ETA) and (ii) an antagonist of the bradykinin B1 receptor (BKB1).

Another aspect of the present invention relates to methods, compounds and composition for preventing and/or treating joint inflammation in a subject. In one embodiment, the method for preventing joint inflammation comprises administering to the subject a combination of (i) an antagonist of the endothelin type A receptor (ETA) and (ii) an antagonist of the bradykinin B1 receptor (BKB1). In a preferred embodiment, the joint inflammation is related to osteoarthritic cartilage degradation.

Yet, an additional aspect of the present invention relates to methods, compounds and composition for treating pain, for preventing osteoarthritic cartilage degradation and/or for preventing joint inflammation, the method comprising administering to the subject a compound of Formula (I) as defined in WO 2006/017938 and/or a compound of Formula (I) as defined in U.S. Pat. No. 7,211,566. In a preferred embodiment, the compound is R-954. In a even preferred embodiment, the compound(s) of Formula (I) is (are) administered in combination with an antagonist of the endothelin type A receptor (ETA) as defined herein.

Further aspects of the invention will be apparent to a person skilled in the art from the following description, claims, and generalizations therein.

### BRIEF DESCRIPTION OF THE FIGURES

In order that the invention may be readily understood, embodiments of the invention are illustrated by way of examples in the accompanying drawings.

FIG. 1 is a panel of macro photographs of rat anterior cruciate transection (ACLT) surgical steps (FIG. 1A to 1L). Photos were taken with a 300-mm macro lens (approximate magnification 2×). This procedure was used to induce experimental OA in laboratory rats.

FIG. 2 is a panel of macro photographs depicting the mode of administration of the compounds in the rat via intra-articular injection into the knee. FIG. 2A: the shaved knee is maintained in extension. FIG. 2B: the needle is inserted under the patellar tendon into the joint space. FIG. 2C: the syringe plunger is depressed slowly. FIG. 2D: successful injection is detected by a momentary swelling of the articular space. Photos were taken with a 300-mm macro lens (approximate magnification 2×).

FIG. 3 depicts the chemical structures of the ETA and BKB1 antagonists used in the examples to prove the effectiveness of treating OA with a dual ETA/BKB1 antagonist approach. Left, BQ-123 (ETA antagonist); right, R-954 (BKB1 antagonist).

FIG. 4 is a panel of photographs depicting the static weight bearing apparatus used to assess joint pain in laboratory rats. Photographs were taken with an animal inside the apparatus, positioned for measurements of the hind limb weight bearing capacity. A, side view; B, angle view; C, front view.

FIG. 5 is a line graph showing that dual antagonist treatment with BQ-123 and R-954 improves static weight bearing tolerance in rats with experimental OA. Static weight bearing was measured repeatedly at defined time points over the course of the study. Data are presented as mean±standard deviation per experimental group (n=4), of weight on the right leg as a percentage of total weight on both hind limbs. Day 0, baseline pre-operative values. Repeated measures analysis of variance with Tukey post-hoc: †, P=0.03115, sham surgery/saline treatment (black diamond) versus ACLT/saline treatment (grey triangle); ‡, P=0.00211, ACLT/BQ-123+R-954 dual treatment (grey circle) versus ACLT/saline treatment (grey triangle).

FIG. 6 is a panel of X-ray pictures, which depict that dual antagonist treatment with BQ-123 and R-954 improves hard-tissue radiological indices of OA in rats. FIG. 6A: no surgery and saline treatment; FIG. 6B: sham surgery and saline treatment; FIG. 6C: ACLT and saline treatment; FIG. 6D: ACLT and BQ-123 treatment; FIG. 6E: ACLT and R-954 treatment; FIG. 6F: ACLT and BQ-123+R-954 dual treatment. T indicates tibial plateau, S indicates subchondral bone, and O indicate osteophytes. Sagittal views. Scale bar, 1 cm.

FIG. 7 is a panel of magnetic resonance images illustrating that dual antagonist treatment with BQ-123 and R-954 improves radiological indices of OA in rats. FIG. 7A: no surgery and saline treatment; FIG. 7B: sham surgery and saline treatment; FIG. 7C: ACLT and saline treatment; FIG. 7D: ACLT and BQ-123 treatment; FIG. 7E: ACLT and R-954 treatment; FIG. 7F: ACLT and BQ-123+R-954 dual treatment. White arrows indicate articular cartilage. Sagittal views. Scale bar, 1 cm.

FIG. 8 is a panel of photomicrographs of histological preparations showing that antagonist treatment with BQ-123 and R-954 protects joint histomorphometry and protects articular type II collagen protein expression in rats with experimental OA. Sagittal sections. Left and middle columns, Safranin O/Fast Green FCF staining. Right column, type II collagen immunohistochemistry. Left column, low-power magnification: scale bar, 200 μm; original magnification 50×. Middle and right columns, high-power magnification: scale bar, 50 μm; original magnification 200×. Conditions by rows: (a), (b), (c) no surgery and saline treatment; (d), (e), (f) sham surgery and saline treatment; (g), (h), (i) ACLT and saline treatment; (j), (k), (I) ACLT and BQ-123 treatment; (m), (n), (o) ACLT and R-954 treatment; (p), (q), (r) ACLT and BQ-123+R-954 dual treatment. In left column: ‘S’ indicates loss of Safranin O staining, ‘N’ indicates cartilage notching, and ‘0’ indicates an osteophyte. In right column: Black arrows indicate type II collagen immunostaining.

FIG. 9 is a panel which depicts the effect of ETA (BQ-123) and BKB1 (R-954) antagonists on MMP-1 protein secretion as detected by Western Blot Human OA chondrocytes were treated with ET-1 for 24 h. Then, specific ETA-antagonist BQ-123 (10 nM), or BKB1 specific receptor antagonist R-954 (10 nM), was added for 30 minutes followed by 24 h of stimulation by BKB1 agonist dKD [des-Arg10]-Kallidin (dKD) (100 nM). Medium was collected for Western Blot detection of secreted MMP-1.

FIG. 10 is a panel of photomicrographs depicting immunohistochemistry. ETA-R and BKB1 dual antagonist (BQ-123+R-954) treatment of ACL-transected rats decreases bradykinin B1 receptor expression (FIG. 10, arrowheads in left panel versus right panel) in rat knee joints.

FIGS. 11A-11D are photomicrographs depicting the effect of BQ-123+R-954 dual inhibition. This effect is inhibitory on COX-2 protein on articular cartilage from rat surgically induced OA. Tissues were obtained from surgery induced OA in a rat model Top panel represent COX-2 immunodetection on tissues from non-treated lesioned animals (FIG. 11A); BQ-123 treatment (FIG. 11B); R-954 treatment (FIG. 11C); BQ-123+R-954 dual treatment (FIG. 11D). Specific immune staining for COX-2 protein is observed as a darker color. Surgery, pharmacological treatment, and tissue collection and processing were performed as described in Kaufman et al., Arthritis Research and Therapy, 2011, 13:R76. FIG. 11E is a bar graph depicting the percentage of COX-2-positive chondrocytes in each of the conditions (mean of 5 slides counted per condition).

### DETAILED DESCRIPTION OF THE INVENTION

### A) General Overview of the Invention

The inventors have discovered the pharmacological significance of the dual targeting of ETA and BKB1 receptors in osteoarthritis, as a local or systemic treatment strategy. ETA and BKB1 dual antagonism improves chronic pain (including joint pain tolerance) and prevents joint degradation in osteoarthritis (including prevention of osteoarthritic cartilage degradation). ETA and BKB1 receptors therefore represent a novel therapeutic target: specific receptor dual antagonism may prove beneficial in disease management. ETA and BKB1 receptors are therefore relevant molecular targets in osteoarthritis, a pathology for which more effective pharmacological interventions are necessary.

The present inventors have also identified compounds that may be useful in the treatment of OA, thereby protecting cartilage tissue from injury, decreasing perceived joint pain, and abrogating joint inflammation.

Therefore, the present invention relates to the control of pain and to compounds, compositions, and their uses for alleviating pain, including, more particularly, increasing tolerance to joint pain. The invention also relates to methods, compounds, and compositions comprising the same for the prevention of osteoarthritic cartilage degradation.

### B) Pharmaceutical Applications

As indicated hereinbefore and exemplified hereinafter, one aspect of the invention concerns methods, compounds and composition for treating pain, e.g. joint pain in a subject in need thereof.

Another aspect of the invention concerns methods, compounds, and composition for preventing osteoarthritic cartilage degradation in a subject in need thereof.

Another aspect of the invention concerns methods, compounds, and composition for treating a subject afflicted by a disease or condition with pain initiated by inflammation.

As used herein, the terms “treatment” or “treating” of a subject includes the application or administration of a suitable compound, or composition of the invention as defined herein to a subject (or application or administration of a compound or composition of the invention to a cell or tissue from a subject) with the purpose of delaying, stabilizing, curing, healing, alleviating, relieving, altering, remedying, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement, remission, slowing disease progression or severity, stabilization, diminishing of symptoms or making the injury, pathology or condition more tolerable to the subject, slowing in the rate of degeneration or decline, making the final point of degeneration less debilitating, or improving a subject's physical or mental well-being. In some embodiments, the term “treating” can include increasing a subject's life expectancy and/or delay before additional treatments are required (e.g. joint replacement surgery).

As used herein, the term “treating pain” includes increasing pain tolerance and/or decreasing perceived pain. In particular embodiments, the methods, compounds and composition of the invention are for increasing pain tolerance and/or for decreasing perceived pain.

As used herein, the term “pain tolerance” refers to the amount of pain that a subject can perceive and withstand before breaking down emotionally and/or physically. Pain tolerance is a concept distinct from pain threshold (the minimum stimulus necessary to produce pain). As used herein, “increasing pain tolerance” generally refers to a situation where a subject can develop a greater pain tolerance (that is, less perceived pain) when compared to a previous state, for instance following administration of suitable compounds and/or compositions, according to the invention, to a subject. Those skilled in the art know how to measure or detect pain tolerance and perceived pain. For instance, it is known that specific instruments, such as a dolorimeter, can be used to detect, measure, and quantify perceived pain. The Exemplification section of the present invention also describes an apparatus for assessing pain tolerance in rats.

As used herein the term “cartilage” refers to the stiff and inflexible connective tissue found in many areas in the bodies of humans and other animals, including the joints between bones, the rib cage, the ear, the nose, the elbow, the knee, the ankle, the bronchial tubes and the intervertebral discs. As used herein, the term “cartilage degradation” refers to the catabolism or the breakdown of the cartilage, including but not limited to degradation of the extracellular matrix (including collagen, proteoglycans and aggrecans) caused by the release of abnormally high levels of degradative enzymes by the joint tissues, mainly from articular cartilage and from synovial membranes. As used herein, the term “osteoarthritic cartilage degradation” refers to degradation of cartilage of the joints, including but not limited to the elbow, the knee, the hand joints (e.g. wrist, fingers, and thumb), the ankle, the foot, the hip, and the intervertebral discs and cartilage of the growth plate.

As used herein the term “subject” includes living organisms susceptible to pain, and more particularly living organisms in which joint-related pain can occur. The term “subject” includes animals such as mammals. Preferably, the subject is a mammal, including, but not limited to, species such as a human, a dog, a cat, a horse, a bovine, a rat, a mouse, and wild animals living in zoos (e.g. lion, tiger, elephant, panda, bear, etc.). More preferably, the subject is a human. Even more preferably, the subject is a human patient in need of treatment, including but not limited to, a human patient having osteoarthritis.

In various embodiments, the subject suffers or is afflicted by a disease or condition including, but not limited to, osteoarthritis, ankylosing spondylitis, joint manifestation of Behçet's disease, fibromyalgia, Ehlers-Danlos syndrome, gout, infectious arthritis, Felty's syndrome, juvenile arthritis, systemic lupus erythematosus, mixed connective tissue disease (MCTD), repetitive stress injury, chronic back injury, and carpal tunnel syndrome.

In other embodiments, the subject may be afflicted by a disease or condition with pain initiated by any manner of inflammation; stimulation of nociceptors in the peripheral nervous system; or by damage to, or malfunction of, the peripheral or central nervous systems; neuropathic pain (caused by damage to or malfunction of any part of the nervous system); or vascular pain (pain from blood vessels); or deep somatic pain (initiated by stimulation of nociceptors in ligaments, tendons, bones, blood vessels, fasciae and muscles: examples include sprains and broken bones); or poorly localized diffuse visceral pain; causalgia (burning pain in the skin of the arms or legs); or chronic pain, that extends beyond the expected period of healing.

As indicated herein, medical and pharmaceutical applications contemplated by the inventors include, but are not limited to, those applications addressing prevention and/or treatment of pain (e.g. increasing pain tolerance/decreasing perceived pain), those applications addressing prevention and/or treatment of osteoarthritic cartilage degradation, and those applications addressing prevention and/or treatment preventing joint inflammation.

As used herein, “preventing” or “prevention” is intended to refer to at least the reduction of likelihood of the risk of (or susceptibility to) acquiring a disease or disorder (i.e. causing at least one of the clinical symptoms of the disease not to develop in a subject that may be exposed to or predisposed to the disease, but does not yet experience or display symptoms of the disease). Biological and physiological parameters for identifying such patients are provided herein and are also well-known by physicians. More particularly, the compounds, compositions, and methods of the invention may be useful in preventing pain (e.g. joint pain), preventing cartilage degradation and/or preventing joint inflammation.

The terms “treatment” or “treating” of a subject includes the application or administration of a suitable compound, or composition of the invention as defined herein to a subject (or application or administration of a compound or composition of the invention to a cell or tissue from a subject) with the purpose of delaying, stabilizing, curing, healing, alleviating, relieving, altering, remedying, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement, remission, slowing disease progression or severity, stabilization, diminishing of symptoms or making the injury, pathology or condition more tolerable to the subject, slowing in the rate of degeneration or decline, making the final point of degeneration less debilitating, or improving a subject's physical or mental well-being. In some embodiments, the term “treating” can include increasing a subject's life expectancy and/or delay before additional treatments are required (e.g. joint replacement surgery).

In various embodiment, the invention concerns administering to a subject in need thereof a combination of both (i) an antagonist of the endothelin type A receptor (ETA) and (ii) an antagonist of the bradykinin B1 receptor (BKB1).

As used herein, the term “antagonist of an Endothelin type A receptor” refers to a compound (natural or synthetic) capable of selectively blocking the binding of endothelin-1 to the endothelin type A receptor (ETA), thereby inhibiting, reducing, or blocking the activity of ETA. Potentially useful examples include, but are not limited to: Bosentan (Ro-470203), Atransentan (ABT627), Tezosentan (Ro-610612), Sitaxsentan (TBC-11251), Darusentan (LU-135252), Clazosentan (Ro61-1790, AXV-034343), ZD-4054, Ambrisentan (LU-208075), TAK-044, Avosentan (SPP301), and BQ-123 (Ihara et al Life Sci 1992, 50(4):247-55). These particular compounds and additional examples are described in Aubert et. al., Expert Opin. Ther. Targets (2009), 13(9): 1069-1084, which is incorporated herein by reference. The chemical structure of most of these compounds in shown in Table A. In a preferred embodiment, the antagonist of the ETA (endothelin type A receptor) is the compound BQ-123 (FIG. 3).

TABLE A
Antagonists of Endothelin type A receptor ETA

| Name | Structure |
|---|---|
| BQ-123 | 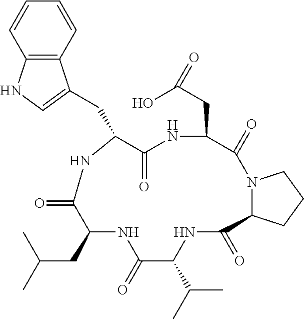 |
| Bosentan | 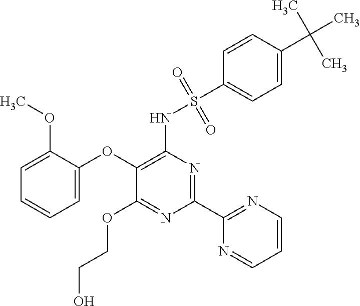 |
| Atrasentan | 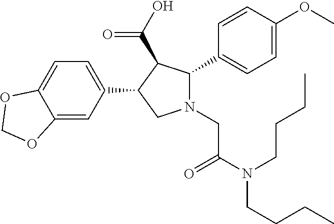 |
| Tezosentan | 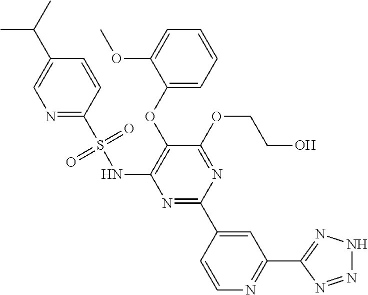 |
| Sitaxsentan | 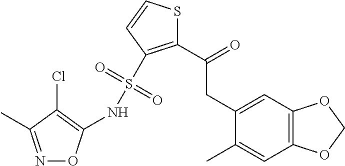 |
| Darusentan | 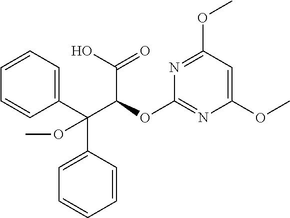 |
| Clazosentan | 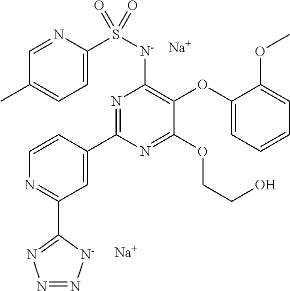 |
| ZD-4054 (Zibotentan) | 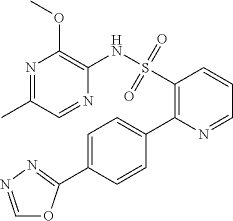 |
| Ambrisentan | 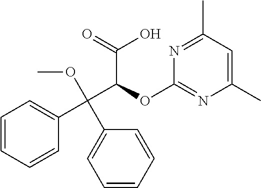 |
| Tak-044 | 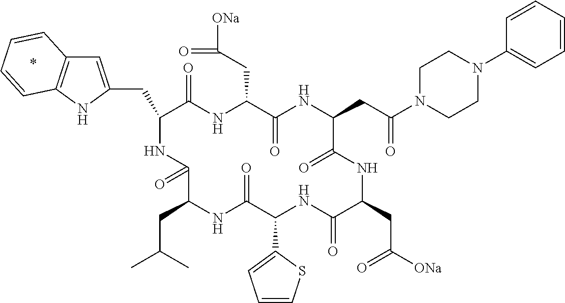 |
| Avosentan | 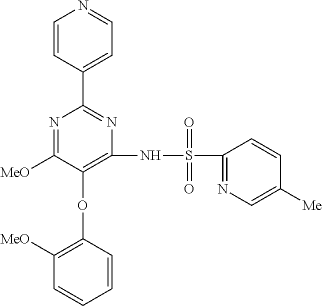 |

As used herein, the term “antagonist of a bradykinin B1 receptor” refers to a compound (natural or synthetic) capable of selectively blocking the binding of bradykinins to the bradykinin B1 receptor and thereby inhibiting, reducing, or blocking the activity of the bradykinin B1 receptor (BKB1). Potentially useful examples include, but are not limited to: MK-0686 (from Merck), SSR-240612 (from Sanofi-Aventis), AMG-379 (from Amgen), R-954 (Gabra B H, Benrezzak O, Pheng L H, Duta D, Daull P, Sirois P, Nantel F, Battistini B: Inhibition of type 1 diabetic hyperalgesia in streptozotocin-induced Wistar versus spontaneous gene-prone BB/Worchester rats: efficacy of a selective bradykinin B1 receptor antagonist. J Neuropathol Exp Neurol 2005, 64(9):782-9 and Neugebauer W, Blais P A, Hallé S, Filteau C, Regoli D, Gobeil F: Kinin B1 receptor antagonists with multi-enzymatic resistance properties. Can J Physiol Pharmacol 2002, 80(4):287-92.), compounds comprising Formula (I) as defined in WO 2006/017938 and compounds of the Formula (I) as defined in U.S. Pat. No. 7,211,566; both patent documents being incorporated herein by reference. These particular compounds and additional examples are described in Fincham et. al., Expert Opin. Ther. Patents (2009), 19(7): 919-941, which is incorporated herein by reference. The chemical structure of some of these compounds in shown in Table B. In a preferred embodiment, the antagonist of bradykinin B1 receptor is the compound R-954 (FIG. 3).

TABLE B
Antagonist of a bradykinin B1 receptor (BKB1)

| Name | Structure |
|---|---|
| R-954 | 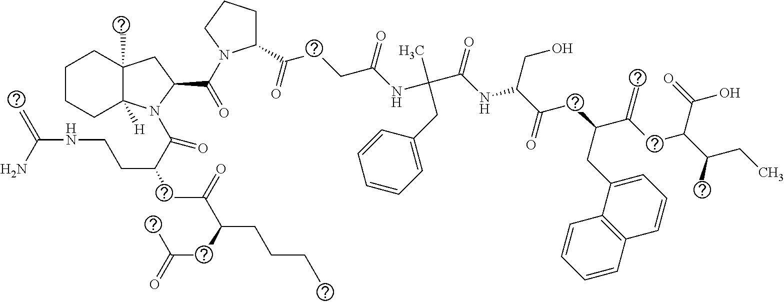 |
| | R-954 |
| MK-0686 | 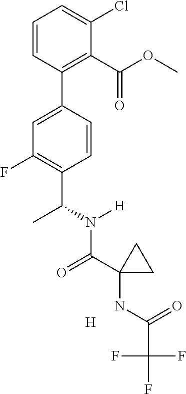 |
| SSR-240612 | 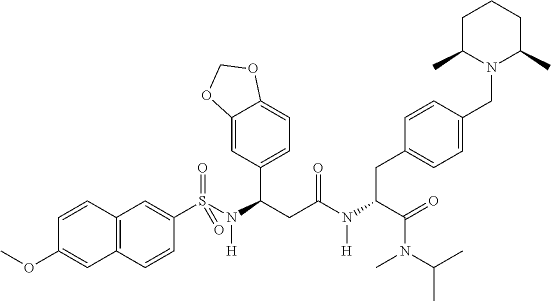 |

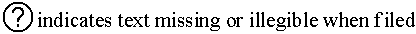

According to the preferred embodiments, the antagonist of the endothelin type A receptor (ETA) and the antagonist of the bradykinin B1 receptor (BKB1) are used in combination. As used herein, the term “combination” refers to a formulation comprising both antagonists, or a mode of administration where both antagonists are administered simultaneously. For instance, the first and second antagonists may be co-administered. As used herein, “combination” also encompasses a mode of administration where both antagonists are administered separately. For instance “combination” encompasses a mode of administration in which the first (or second) antagonist is administered, wherein second (or first) antagonist may have been previously administered. The administration of both antagonists may also be executed step-wise by the same or by different actors. For example, one actor may administer to a subject a first antagonist, and a second actor may administer to the subject a second antagonist. The administering steps may be executed at the same time, or nearly the same time, or at distant times, so long as the antagonists are capable of combined biological activity on the subject. In preferred embodiment, the combination is a synergistic combination such that a cooperative interaction exists between both antagonists and that their combined effect is greater than the sum of their individual effects.

In a particular embodiment, the antagonist of the endothelin type A receptor (ETA) consists of compound BQ-123, or a pharmaceutically acceptable salt thereof (structure shown in FIG. 3) and the antagonist of the bradykinin B1 receptor (BKB1) consists of compound compound R-954, or a pharmaceutically acceptable salt thereof (structure also shown in FIG. 3).

Of course, the class(es) of compounds comprised by the invention may be combined with additional active agents useful for decreasing joint pain and/or for preventing osteoarthritic cartilage degradation, including, but not limited to, analgesics, corticosteroids, hyaluronan, antibodies, and so forth (reviewed in: Recommendations for the medical management of osteoarthritis of the hip and knee: 2000 update. American College of Rheumatology Subcommittee on Osteoarthritis Guidelines. Arthritis Rheum (2000) vol. 43 (9) pp. 1905-15).

### C) Pharmaceutical Compositions and Formulations

Related aspects of the invention concern pharmaceutical compositions and medicaments comprising an antagonist of the endothelin type A receptor (ETA) combined with an antagonist of the bradykinin B1 receptor (BKB1). Another aspect concerns pharmaceutical compositions and medicaments comprising a compound of Formula (I) as defined in WO 2006/017938 and/or a compound of Formula (I) as defined in U.S. Pat. No. 7,211,566. As indicated hereinbefore, the compounds of the invention may be useful in treating pain, preventing osteoarthritic cartilage degradation and pathological joint tissue changes, diminishing OA joint inflammation, or any combination of these effects.

In preferred embodiments, therapeutically effective amounts of the compounds of the invention are being used. As used herein, the term “therapeutically effective amount” means the amount of a compound that, when administered to a subject for treating or preventing a particular disorder, disease or condition, is sufficient to effect such treatment or prevention of that disorder, disease or condition. Dosages and therapeutically effective amounts may vary depending upon many factors, including the half-life and activity of the specific agent employed, the age, body weight, general health, gender, and diet of the subject, the time of administration, the route of administration, the rate of excretion, and any drug combination the effect which the practitioner desires the conjugate chemicals (if any) to have upon the subject and the properties of the compound (e.g. bioavailability, stability, potency, toxicity, etc), and the particular disorder(s) the subject is suffering from. In addition, the therapeutically effective amount may depend on the subject's blood parameters (e.g. lipid profile, insulin levels, glycemia), the severity of the disease state, organ function, or underlying disease or complications. Such appropriate doses may be determined using any available assays including the assays described herein. When one or more of the compounds of the invention are to be administered to humans, a physician may for example, prescribe a relatively low dose at first, subsequently increasing the dose until an appropriate response is obtained.

As used herein, the term “pharmaceutical composition” refers to the presence of one or more compounds of the present invention as defined herein and at least one pharmaceutically acceptable vehicle. An example of a compound of the present invention is an ETA antagonist as defined herein (e.g. BQ-123). Another example of a compound of the present invention is a BKB1 antagonist as defined herein (e.g. R-954).

“Pharmaceutically acceptable vehicle” refers to a diluent, adjuvant, excipient, or carrier with which a compound is administered. The term “pharmaceutically acceptable” refers to drugs, medicaments, inert ingredients etc., which are suitable for use in contact with the tissues of humans and lower animals without undue toxicity, incompatibility, instability, irritation, allergic response, and the like, commensurate with a reasonable benefit/risk ratio. It preferably refers to a salt, chemical conjugate, or other composition that is approved or approvable by a regulatory agency of the Federal or State governments, or listed in the U.S. Pharmacopoeia or other generally recognized pharmacopoeia for use in animals and more particularly in humans. The pharmaceutically acceptable vehicle can be a solvent or dispersion medium containing one or more of the following chemicals: water, ethanol, polyol (for example, glycerol, propylene glycol, and liquid polyethylene glycol), suitable mixtures thereof, and vegetable oils. Additional examples of pharmaceutically acceptable vehicles include, but are not limited to, Water for Injection; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Prevention of the action of microorganisms can be achieved by addition of various antibacterial and antifungal agents, for example, parabens, chlorobutanol, phenol, ascorbic acid, thimerosal, and the like. In many cases, isotonic agents are included in the composition, for example, sugars, sodium chloride, or polyalcohols such as mannitol and sorbitol Prolonged absorption of injectable compositions can be brought about by including an agent which delays absorption, for example, aluminum monostearate or gelatin.

In one embodiment, the compositions of the present invention may be manufactured by mixing an antagonist of the endothelin type A receptor (ETA) and an antagonist of the bradykinin B1 receptor (BKB1) with a pharmaceutically acceptable vehicle.

In some embodiments, the compositions of the invention comprise an effective amount of a compound of the Formula (I) as defined in WO 2006/017938 and/or a compound of the Formula (I) as defined in U.S. Pat. No. 7,211,566.

In some embodiments, the invention pertains to pharmaceutical compositions for treating pain (e.g. decreasing perceived joint pain and/or increasing joint pain tolerance), preventing osteoarthritic cartilage degradation, and/or diminishing joint inflammation in a subject in a subject in need thereof.

The compounds of the invention may be formulated prior to administration into pharmaceutical compositions using available techniques and procedures. For instance, the pharmaceutical compositions may be formulated into suitable administration (orally, parenterally, (intravascular (IV), intra-arterial (IA), intramuscular (IM), depo-IM, subcutaneous (SC), and depo-SC), sublingually, intranasally (inhalation), intrathecally, topically, or rectally. Preferred routes are those allowing a local treatment of the pain and/or inflammation, for instance locally at a site where the subject feels pain. More preferably the pharmaceutical compositions are formulated for a local injection in a human subject at a site of pain, osteoarthritic cartilage degradation, and/or joint inflammation.

In a preferred embodiment, the compounds of the invention are administered via intra-articular injection to the affected joint. A skilled practitioner, such as a physician or nurse practitioner, can carry out intra-articular injection in a manner of minutes with minimal patient discomfort. Intra-articular injection is the preferred mode of treatment for OA because articular cartilage is avascular and is nourished by diffusion. Some compounds, (e.g. peptide antagonists) have short circulating half-lives and/or can be degraded by gastrointestinal passage or by blood circulation. Thus, local treatment may ensure that the compounds reach the target tissues and acts with maximum efficacy, while minimizing any systemic side effects of treatment.

Formulations and pharmaceutical compositions according to the invention may conveniently be presented in unit dosage form and may be prepared by any methods well-known in the art of pharmacy. Methods of preparing these formulations or compositions include the step of bringing into association a compound of the present invention with a pharmaceutically acceptable vehicle (e.g. an inert diluent or an injectable carrier) and, optionally, one or more accessory ingredients. In general, the formulations are prepared by uniformly and intimately bringing into association a compound of the present invention with liquid carriers, or finely divided solid carriers, or both, and then, if necessary, shaping the product. The amount of the therapeutic agent in such therapeutically useful compositions is such that a suitable dosage will be obtained.

Formulations and pharmaceutical compositions of the invention for oral uses may be in the form of capsules (e.g. hard or soft shell gelatin capsule), cachets, pills, tablets, lozenges, powders, granules, pellets, dragees, e.g., coated (e.g., enteric coated) or uncoated, or as a solution or a suspension in an aqueous or non-aqueous liquid, or as an oil-in-water or water-in-oil liquid emulsion, or as an elixir or syrup, or as pastilles or as mouth washes and the like, each containing a predetermined amount of a compound of the present invention as an active ingredient. A compound of the present invention may also be administered as a bolus, electuary, or paste. Moreover, in certain embodiments these formulations can be formulated to (a) provide for instant or rapid drug release (i.e., have no coating on them); (b) be coated, e.g., to provide for sustained drug release over time; or (c) be coated with an enteric coating (e.g. for better gastrointestinal tolerability).

Pharmaceutical compositions suitable for injectable use may include sterile aqueous solutions (where water soluble) or dispersions and sterile powders for the extemporaneous preparation of sterile injectable solutions or dispersions. In all cases, the injectable composition must be sterile and must be fluid to the extent that easy syringability exists. It must be stable under the conditions of manufacture and storage and must be preserved against the contaminating action of microorganisms such as bacteria and fungi. Sterile injectable solutions can be prepared by incorporating the therapeutic agent in the required amount in an appropriate solvent with one or a combination of ingredients enumerated above, as required, followed by filtered sterilization. Generally, dispersions are prepared by incorporating the therapeutic agent into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient (i.e. the therapeutic agent) plus any additional desired ingredient from a previously sterile-filtered solution thereof.

The compositions of this invention may also be administered topically to a subject, e.g., by the direct laying on or spreading of the composition on the epidermal or epithelial tissue of the subject, or transdermally via a “patch”. Such compositions include, for example, lotions, creams, solutions, gels and solids. Suitable carriers for topical administration typically remain in place on the skin as a continuous film, and resist being removed by perspiration or immersion in water. Generally, the carrier is organic in nature and capable of having dispersed or dissolved therein the therapeutic agent. The carrier may include pharmaceutically acceptable emollients, emulsifiers, thickening agents, solvents and the like.

Those skilled in the art will recognize, or be able to ascertain, using no more than routine experimentation, numerous equivalents to the specific procedures, embodiments, claims, and examples described herein. Such equivalents are considered to be within the scope of this invention, and covered by the claims appended hereto. The invention is further illustrated by the following example, which should not be construed as further or specifically limiting.

### EXAMPLES

The Examples set forth herein below provide exemplary methods and results showing that dual antagonism of endothelin type A and bradykinin B1 receptors improves pain tolerance and protects joint morphology in a surgical model of osteoarthritis. Also provided are exemplary methods for assaying the compounds of the invention for in vivo efficacy.

### Example 1

Summary: Endothelin-1, the vasoconstrictor peptide, influences cartilage metabolism mainly via endothelin receptor type A (ETA). Along with the inflammatory nonapeptide vasodilator bradykinin (BK), which acts via bradykinin receptor B1 (BKB1) in chronic inflammatory conditions, these vasoactive factors potentiate joint pain and inflammation. This example describes a preclinical animal study of the efficacy of treatment of surgically induced osteoarthritis with ETA and/or BKB1 specific peptide antagonists. The results show that antagonism of both receptors diminishes osteoarthritis progress and joint pain in a synergistic manner.

Materials and Methods

Rat Model of Osteoarthritis

Animals

Eight-week-old male Lewis rats were purchased from Charles River Canada (Saint-Constant, QC) and housed under standard conditions. All procedures were approved by the Sainte-Justine Hospital Research Centre animal ethics committee and conformed to Canadian Council on Animal Care guidelines.

Study Design

The study was conducted as a fractional factorial experiment. Animals were randomly assigned to one of three surgery conditions: anterior cruciate ligament transection (ACLT), sham surgery, or no surgery (negative control). Subsequently, animals were assigned to one of four treatment groups, as detailed below (Table 1). Six experimental groups were designated in the fractional factorial study, with 4 subjects per group.

TABLE 1
Experimental groups

| Group Number | Surgery | Treatment |
|---|---|---|
| 1 | None | Saline |
| 2 | Sham | Saline |
| 3 | ACLT | Saline |
| 4 | ACLT | BQ-123 |
| 5 | ACLT | R-954 |
| 6 | ACLT | BQ-123 + R-954 |

Surgical Technique

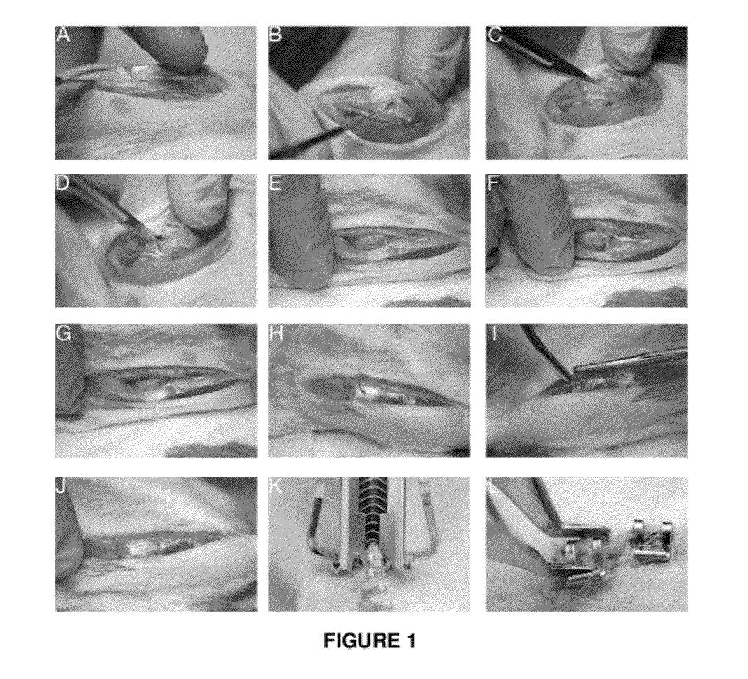

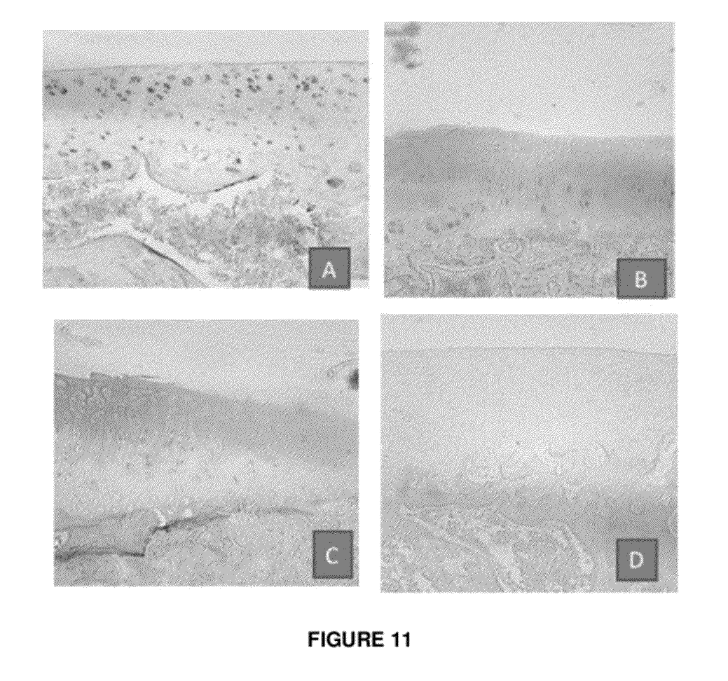

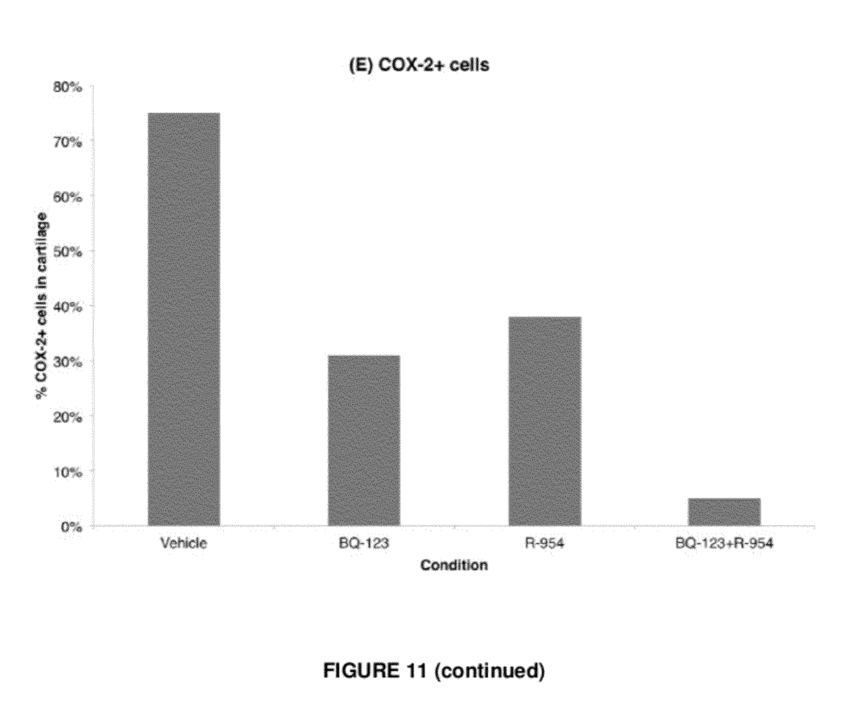

OA was induced by surgical transection of the right anterior cruciate ligament. The procedure was modified from previously published reports [Appleton C T G, McErlain D D, Pitelka V, Schwartz N, Bernier S M, Henry J L, Holdsworth D W, Beier F: Forced mobilization accelerates pathogenesis: characterization of a preclinical surgical model of osteoarthritis. Arthritis Res Ther 2007, 9:R13. Hayami T, Pickarski M, Zhuo Y, Wesolowski G A, Rodan G A, Duong L T: Characterization of articular cartilage and subchondral bone changes in the rat anterior cruciate ligament transection and meniscectomized models of osteoarthritis. Bone 2006, 38(2):234-43. Stoop R, Buma P, van der Kraan P M, Hollander A P, Billinghurst R C, Meijers T H, Poole A R, van den Berg W B: Type II collagen degradation in articular cartilage fibrillation after anterior cruciate ligament transection in rats. Osteoarthr Cartil 2001, 9(4):308-15. Williams J M, Felten D L, Peterson R G, O'Connor B L: Effects of surgically induced instability on rat knee articular cartilage. J Anat 1982, 134(Pt 1):103-9.]. Animals were anaesthetized with inhaled isoflurane (3%/L O2 induction in chamber, 2%/L O2 maintenance with face-mask), and prepared for surgery by clipping the hair over the ventral and medial aspects of the right leg from hindpaw to hip. The skin was disinfected with povidone-iodine, and a 3-cm incision was made medial to the patellar tendon (FIG. 1A). The subcutaneous tissue and muscle were then incised and the patella laterally sublaxed; the joint capsule was opened with the limb hyperextended (FIG. 1B). With the limb in full flexion, the anterior cruciate ligament was visualized by blunt dissection, and sectioned by a latero-medial cut parallel to the tibial plateau, using a #11 scalpel blade (FIG. 1C-D). Transection was confirmed with the anterior drawer test (FIG. 1E-F: E depicts the knee before, and F shows an anterior drawer). The patella was then replaced, and the limb extended (FIG. 1G). The joint capsule (FIG. 1H) and muscle layers (FIG. 11) were closed with 4-0 polygalactin absorbable suture (horizontal mattress stitch, FIG. 1J). 50 μL of lidocaine was then injected into the joint capsule to provide local analgesia. Skin was closed with steel surgical staples (FIG. 1K). Post-operative hydration (6 mL/kg saline) and systemic analgesia (0.1 mg/kg buprenorphine HCl) were provided by subcutaneous injection. Surgical staples were removed 14 days post-operatively (FIG. 1L). Sham surgery consisted of all of the above except ligament transection.

Drug Treatment

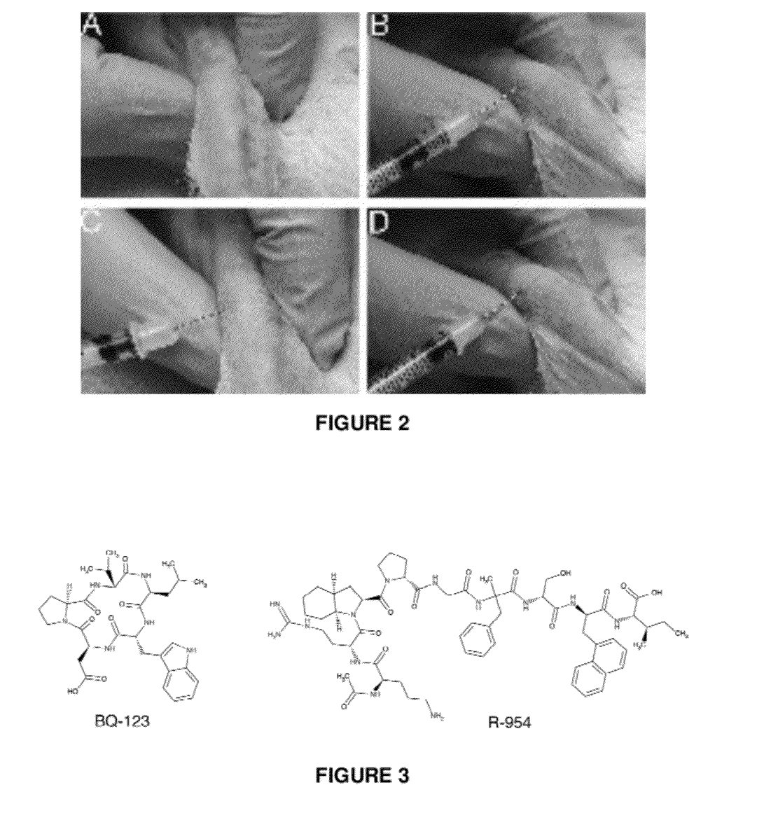

Over the course of two months post-operatively, animals were treated by weekly intra-articular injections of ETA and/or BKB1 specific peptide antagonists: BQ-123 (ETA antagonist; Sigma-Aldrich, Oakville, ON) [Ihara M, Noguchi K, Saeki T, Fukuroda T, Tsuchida S, Kimura S, Fukami T, Ishikawa K, Nishikibe M, Yano M: Biological profiles of highly potent novel endothelin antagonists selective for the ETA receptor. Life Sci 1992, 50(4):247-55; Ihara M, Ishikawa K, Fukuroda T, Saeki T, Funabashi K, Fukami T, Suda H, Yano M: In vitro biological profile of a highly potent novel endothelin (ET) antagonist BQ-123 selective for the ETA receptor. J Cardiovasc Pharmacol 1992, 20 Suppl 12:S11-4.], R-954 (BKB1 antagonist; kind gift from Pierre Sirois, IPS Thérapeutique, Sherbrooke, QC) [Gabra B H, Benrezzak O, Pheng L H, Duta D, Daull P, Sirois P, Nantel F, Battistini B: Inhibition of type 1 diabetic hyperalgesia in streptozotocin-induced Wistar versus spontaneous gene-prone BB/Worchester rats: efficacy of a selective bradykinin B1 receptor antagonist. J Neuropathol Exp Neurol 2005, 64(9):782-9; Neugebauer W, Blais P A, Hallé S, Filteau C, Regoli D, Gobeil F: Kinin B1 receptor antagonists with multi-enzymatic resistance properties. Can J Physiol Pharmacol 2002, 80(4):287-92.], both, or saline vehicle, was injected into the right knee at a dose of 30 nmol in a volume of 50 μL. Injections were performed under isoflurane anaesthesia, using a 28 G needle (FIG. 2). Chemical structures of the antagonists are depicted in FIG. 3.

### Static Weight Bearing

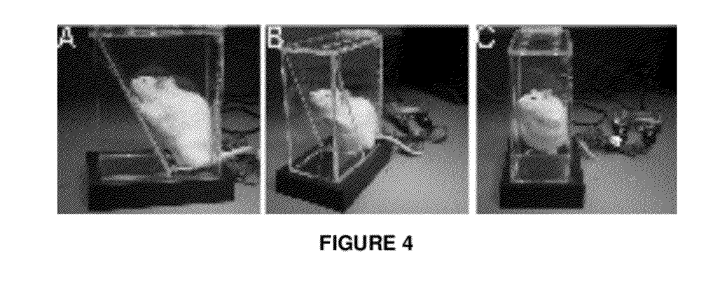

Over the course of the study, knee joint pain was evaluated biweekly by the static weight bearing test. A static weight bearing apparatus was reverse-engineered from previously published reports [Bove S E, Calcaterra S L, Brooker R M, Huber C M, Guzman R E, Juneau P L, Schrier D J, Kilgore K S: Weight bearing as a measure of disease progression and efficacy of anti-inflammatory compounds in a model of monosodium iodoacetate-induced osteoarthritis. Osteoarthr Cartil 2003, 11(11):821-30 Bove S E, Laemont K D, Brooker R M, Osborn M N, Sanchez B M, Guzman R E, Hook K E, Juneau P L, Connor J R, Kilgore K S: Surgically induced osteoarthritis in the rat results in the development of both osteoarthritis-like joint pain and secondary hyperalgesia. Osteoarthr Cartil 2006, 14(10):1041-8 Vermeirsch H, Biermans R, Salmon P L, Meert T F: Evaluation of pain behavior and bone destruction in two arthritic models in guinea pig and rat. Pharmacol Biochem Behav 2007, 87(3):349-59.], designed, and machined by Usinage FB (Le Gardeur, Quebec). After conditioning, animals were introduced to the apparatus and restrained in a Plexiglas chamber with an angled base, such that each hind paw rested on a separate force plate connected to a load cell (FIG. 4). The weight in grams distributed on each hind limb was recorded by a computer software interface (Futek™ USB software interface version 2.10). Data were transferred off-line to a personal computer, and the weight bearing on the right hind limb as a percentage of total weight bearing on both hind limbs was calculated by the following equation [Pomonis J D, Boulet J M, Gottshall S L, Phillips S, Sellers R, Bunton T, Walker K: Development and pharmacological characterization of a rat model of osteoarthritis pain. Pain 2005, 114(3):339-46.]:

$$\% weight on right leg = \frac{weight on right leg}{weight on right leg + weight on left leg} \times 100$$

Measurements were averaged over three 30-second test periods. All values are given as mean±standard deviation.

Static weight bearing was analyzed by repeated measures analysis of variance (ANOVA) [Ware J H Linear models for the analysis of longitudinal studies. The American Statistician, 39(2):95-101, 1985; Louis T A: General methods for analysing repeated measures. Stat Med, 7 (1-2):29-45, 1988.]. Test values were taken as the dependent variable and treatment group as the independent variable, with the animal as the grouping factor. Sphericity was confirmed with Mauchly's W test. Tukey multiple comparisons testing was used to establish significance in between groups, with directionality taken from the sign of the mean difference. P-values less than 0.05 were considered statistically significant. Analyses were conducted using R (version 2.11.0) [R Development Core Team: R: A Language and Environment for Statistical Computing. Vienna, Austria: R Foundation for Statistical Computing 2010.].

Euthanasia and Sample Preparation

At four or eight weeks post-surgery, animals were sacrificed by cardiac puncture under deep isoflurane anaesthesia. The right knee was dissected, and 40-mm-long samples were cut and stored in phosphate-buffered saline until scanned by digital micro-X-ray (DX) and/or micro-magnetic resonance (MR) imaging. Samples were dissected the same day as the radiological scans.

Digital Micro-X-Ray

All knee samples were X-rayed using a Faxitron MX-20™ specimen X-ray system (Faxitron X-Ray Corporation, Lincolnshire, Ill.). Anteroposterior and lateral views were acquired at 5× magnification (10×10 μm pixel size) using a dose of 26 kV for 6 seconds. Images were analyzed using OsiriX™ software (version 3.7.1) [Rosset et al. OsiriX: an open-source software for navigating in multidimensional DICOM images. Journal of digital imaging (2004) 17(3): 205-16]. Radiological evidence of joint degradation, such as joint space narrowing and osteophyte formation, was evaluated. Radiological evidence of joint degradation was scored by two blinded examiners using an OA radiological score modified from Clark et al. [Clark R L, Cuttino JT Jr, Anderle S K, Cromartie W J, Schwab J H: Radiologic analysis of arthritis in rats after systemic injection of streptococcal cell walls. Arthritis Rheum 1979, 22:25-35.] and Esser et al. [Esser R E, Hildebrand A R, Angelo R A, Watts L M, Murphey M D, Baugh L E: Measurement of radiographic changes in adjuvant-induced arthritis in rats by quantitative image analysis. Arthritis Rheum 1995, 38:129-38.]. Bone demineralization, subchondral bone erosion, and heterotopic ossification were all scored on a scale from zero (normal) to three (marked degenerative changes). Total scores were calculated by summing the individual scores for each index, with a maximum possible score of nine.

OA radiological scores were statistically analyzed by one-way ANOVA, with total scores taken as the dependent variable and treatment group as the independent variable. Pairwise post-hoc testing with Holm correction was used to establish significance in between treatment groups. P-values less than 0.05 were considered statistically significant. Analyses were conducted using R (version 2.11.0) [R Development Core Team: R: A Language and Environment for Statistical Computing. Vienna, Austria: R Foundation for Statistical Computing 2010.].

Micro-Magnetic Resonance Imaging

Image Acquisition

A subset of animals were sacrificed four weeks post-operatively and their right knees were imaged by micro-MR. Imaging was performed using a Bruker PharmaScan™ (Ettlingen, Germany) 7 Tesla MR scanner at the McGill University Small Animal Imaging Lab (Montreal, QC). Knee samples were placed in a custom-made support inside a 15-mL centrifuge tube, which was then filled with the MR-inert buffer FC-770™ (3M Fluorinert™ Electronic Liquid). Samples were introduced into a 1H mouse brain radio frequency (RF) coil (inner diameter 22 mm), and centred in the magnet. The RF coil was tuned and matched to the sample, and the magnet was then shimmed. The system was controlled via Bruker ParaVision™ software (version 5.0).

Positioning was confirmed with a tri-pilot rapid scan, which was then used to place 14 coronal slices for two-dimensional anatomical scanning of the joint using a rapid acquisition with relaxation enhancement (RARE) multiecho spin echo pulse sequence (TurboRARE). Scan parameters were as follows: repetition time (TR)=3500 ms, echo time (TE)=36 ms, echo train length (ETL)=8, slice thickness=500 μm, acquisition matrix=384×384, and number of averages=4. Voxel size was 104.16×104.16×500 μm. These scans were then repeated in the sagittal projection.

Once these scans were acquired, one 1-mm-thick axial slice was placed in the centre of the knee joint in order to scan the articular cartilage with a series of multislice multiecho (MSME) T2-weighted pulse sequences. Scan parameters were TR=3500 ms, ETL=1, acquisition matrix=192×256, with voxel size of 156.25×156.25×1000 μm. 16 different TE were used: 10, 20, 30, 40, 50, 60, 70, 80, 90, 100, 110, 120, 130, 140, 150, and 160 ms. Total scan time was roughly 1 hour per sample. Scan sequences were based on previously published reports [Chou M C, Tsai P H, Huang G S, Lee H S, Lee C H, Lin M H, Lin C Y, Chung H W: Correlation between the MR T2 value at 4.7 T and relative water content in articular cartilage in experimental osteoarthritis induced by ACL transection. Osteoarthr Cartil 2009, 17(4):441-7.].

Image Processing and Analysis

After acquisition, images were analyzed using OsiriX software (version 3.7.1). Anatomical TurboRARE images were examined for correct depiction of anatomical features of the knee joint, and to confirm ACLT where applicable. As well, images were analyzed for signs of cartilage decay, indicated by lower signal intensity of the articular surfaces. The MSME-T2 images were aligned into an image stack, and regions of interest, corresponding to the articular cartilage, were manually drawn and propagated throughout the stack. A mean T2 fit map was then automatically generated by fitting the signal intensity to the spin-spin relaxation signal decay equation

$$S ( TE ) = {M}_{0} exp - \frac{TE}{T 2}$$

where signal intensity S is defined as a function of echo time TE, and is related to the spin density M0 and the transverse relaxation time T2. The equation was solved for the mean T2 value over the 16-image stack by using least-squares single-exponential curve-fitting, with initial guesses of M0=signal intensity at 10 ms and T2=30 ms, in order to guarantee rapid convergence [Chou M C, Tsai P H, Huang G S, Lee H S, Lee C H, Lin M H, Lin C Y, Chung H W: Correlation between the MR T2 value at 4.7 T and relative water content in articular cartilage in experimental osteoarthritis induced by ACL transection. Osteoarthr Cartil 2009, 17(4):441-7.]. OsiriX™ then generated a T2 fit map graph with regression line and values for T2 and M0.

Cartilage and Bone Histomorphology

After radiological examination, knee samples were fixed in 10% neutral buffered formalin for two weeks, decalcified with RDO Rapid Decalcifier™ (Apex Engineering Products, Aurora, Ill.) for three days, circulated, and embedded in paraffin. Five-micron sagittal sections were acquired from the middle of the knee joint. Histomorphological staining was performed as previously described [Rosenberg L: Chemical basis for the histological use of safranin O in the study of articular cartilage. J Bone Joint Surg Am 1971, 53:69-82]: slides were deparrafinized, rehydrated, stained with Safranin O (which colours proteoglycans red) and Fast Green FCF (which colours collagens green), counterstained with hematoxylin, dehydrated, cleared, and mounted in Permount™. Representative digital photomicrographs were acquired with a Leica DM R™ microscope (Wetzlar, Germany) fitted with a QImaging Retiga 1300 B™ camera (Surrey, BC), controlled by QCapture™ software (version 2.95.0). All images were captured at 50× magnification and subsequently colour-matched and balanced using Adobe Photoshop CS3™.

Histopathological Scoring

Four slides from each condition were scored by two blinded examiners using the Osteoarthritis Research Society International (OARSI) histopathology assessment system [Pritzker K P H, Gay S, Jimenez S A, Ostergaard K, Pelletier J P, Revell P A, Salter D, van den Berg W B: Osteoarthritis cartilage histopathology: grading and staging. Osteoarthr Cartil 2006, 14:13-29.], which assigns numeric values to grade, or depth progression into cartilage (0-6), and stage, or extent of joint involvement (0-4); multiplying grade and stage yields a total OA score with a maximum value of 24. Scores were averaged in between the two examiners; inter-examiner variation was within ±5%.

OARSI scores were statistically analyzed by one-way ANOVA, with total scores taken as the dependent variable and treatment group as the independent variable. Pairwise post-hoc testing with Holm correction was used to establish significance in between treatment groups. P-values less than 0.05 were considered statistically significant. Analyses were conducted using R (version 2.11.0) [R Development Core Team: R: A Language and Environment for Statistical Computing. Vienna, Austria: R Foundation for Statistical Computing 2010.]

Immunohistochemistry

Additional 5-micron sections were processed for immunohistochemical detection of type II collagen. Slides were deparaffinized, rehydrated, and washed in phosphate-buffered saline (PBS). Sections were incubated in 2 mg/mL hyaluronidase for 30 minutes at 37° C., followed by permeabilization with 0.3% Triton X-100 for 30 minutes at room temperature. Endogenous peroxidase activity was then quenched with 2% hydrogen peroxide in PBS for 15 minutes. Sections were blocked with normal mouse serum (Vector Laboratories, Burlingame, Calif.) for 1 hour, after which they were blotted and then incubated with monoclonal mouse anti-rat type II collagen (clone SPM239; Spring Bioscience, Pleasanton, Calif.) for 18 hours at 4° C. Sections were then washed in PBS, incubated with biotinylated anti-mouse IgG (Vector) for 1 hour at room temperature, and stained using the avidin-biotin complex method (Vectastain ABC™ kit; Vector). Color was developed using 3,3′-diaminobenzidine (Dako Diagnostics, Mississauga, Ontario) containing hydrogen peroxide. Slides were counterstained with Harris modified hematoxylin, dehydrated, cleared, mounted, and examined by light microscopy as described above.

Results

Dual Antagonism Ameliorates OA Pain Tolerance

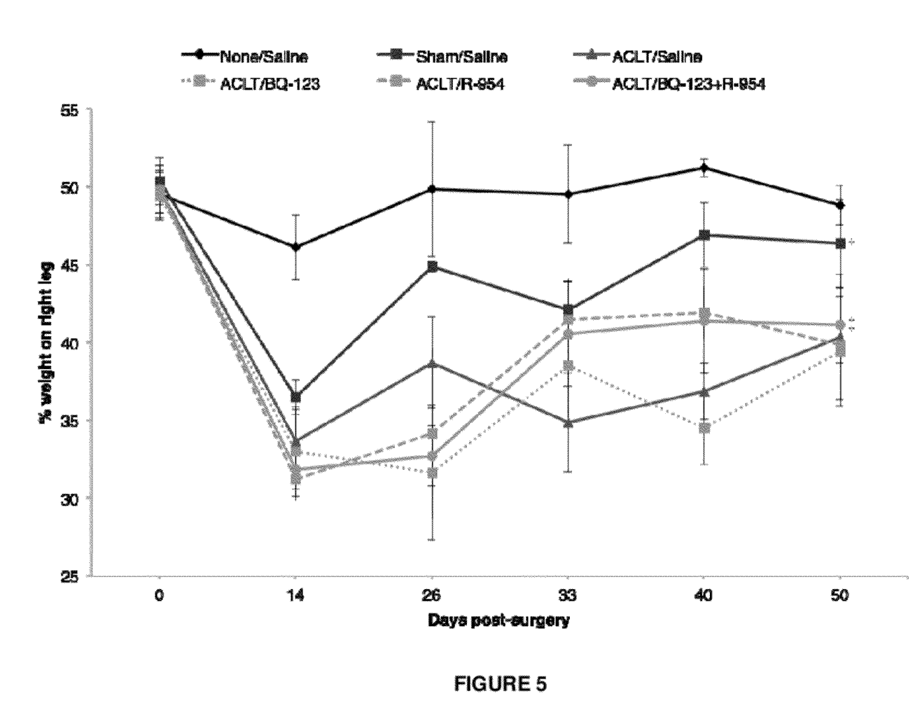

To determine the effects of ETA and/or BKB1 local antagonist treatment on nociception in a surgical OA model, the static weight bearing asymmetry of the animals was measured repeatedly over the course of the study (FIG. 5). Pre-operative baseline values for all groups indicated hind limb weight bearing symmetry (49.89±0.42%). Unoperated vehicle-treated animals showed no important changes in hind limb weight bearing from baseline pre-operative values over the course of the study, staying roughly within ±4% of even weight distribution. Sham-operated vehicle-treated animals displayed an initial weight bearing imbalance 14 days post-operatively (36.47±1.12%), but recovered weight bearing symmetry quickly thereafter (44.84±0.33% by day 26 post-operatively). ACLT vehicle-treated animals showed significant weight bearing imbalance two weeks post-operatively, down to 33.66±2.05% weight on the right leg, suggesting severe nociception. All animals had similar nociceptive tolerance at the last measured time-point (day 50 post-operatively), indicating nociceptive adaptation, but drug-treated animals were able to recover faster than saline-treated animals (up to 40.54±3.36% weight on right leg by day 33 post-operatively).

Repeated measures analysis of variance of the static weight bearing data, followed by Tukey post-hoc hypothesis tests, demonstrated that treatment with both BQ-123 and R-954 significantly ameliorated pain tolerance in ACLT animals over the study period, as compared to saline-treated positive controls (P=0.00211; FIG. 5). Sham surgery was found to be slightly less painful than ACL transection (P=0.03115; FIG. 5), confirming that ACLT is necessary for a maximal nociceptive response. Furthermore, nociception in the sham-operated animals was comparable to unoperated animals, with no statistically significant difference calculated (P=0.99313).

Dual Antagonist Treatment Improves Radiological Indices of OA

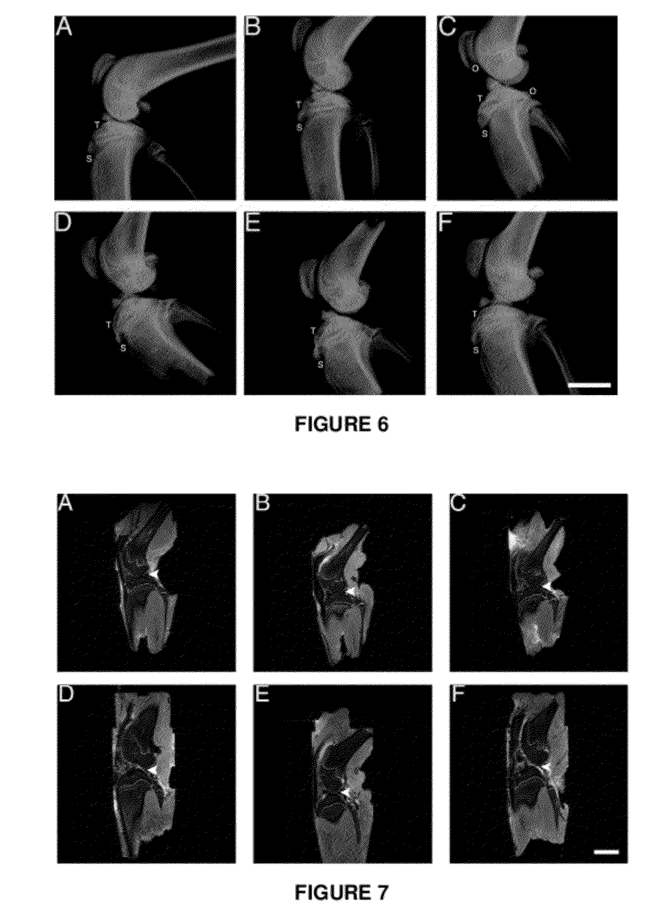

Right knee joints were dissected at the end of the study period and imaged by DX (FIG. 6) and MR (FIG. 7) to examine the radiological effects of antagonist treatments. ACLT rapidly induced radiological evidence of OA: knee joints showed signs of degradation such as subchondral bone remodelling, osteophyte formation (FIG. 6C), cartilage layer thinning (FIG. 6C), and lengthened cartilage T2 (Table 2). Sham surgery and/or intra-articular injection, in and of themselves, did not significantly affect joint radiomorphology (FIG. 6A-B and FIG. 7A-B). DX analysis of antagonist-treated knee joints showed less subchondral bone remodelling, fewer osteophytes, and larger joint space width than saline-treated animals (FIG. 6D-F). Dual ETA/BKB1 antagonism appeared to be more protective than single antagonism: less subchondral bone remodelling and greater trabecular integrity was observed in the dual-antagonist-treated animals than in the single-antagonist-treated animals. Radiological scoring of the DX views for a panel of OA joint degenerative changes (Table 3) demonstrated that treatment with BQ-123, R-954, or both, significantly ameliorated radiological indices of disease progression in ACLT animals, as compared to saline-treated positive controls (0.0020≦P≦0.0214, one-way ANOVA with Holm post-hoc). MR analysis of knee joints revealed that antagonist-treated animals had greater cartilage thickness and fewer cartilage lesions (FIG. 7D-F), as well as shorter cartilage T2 (Table 2) than saline-treated animals. These data suggest that antagonist treatment protected joint radiomorphology after ACLT.

TABLE 2
Cartilage mean T2 values

| | | | Mean T2 value |
|---|---|---|---|
| Group Number | Surgery | Treatment | (ms) * |
| 1 | None | Saline | 51.60 |
| 2 | Sham | Saline | 52.12 |
| 3 | ACLT | Saline | 63.23 |
| 4 | ACLT | BQ-123 | 61.13 |
| 5 | ACLT | R-954 | 64.38 |
| 6 | ACLT | BQ-123 + R-954 | 56.57 |

* Cartilage mean T2 values were calculated for all conditions using OsiriX ™ software (version 3.7.1). Statistical significance in between groups was not achieved.

TABLE 3
OA radiological scores

| | | | Mean | |
|---|---|---|---|---|
| | | | radiological | Standard |
| Group Number | Surgery | Treatment | score* | deviation |
| 1 | None | Saline | 0.25 | 0.50 |
| 2 | Sham | Saline | 1.16 | 0.75 |
| 3 | ACLT | Saline | 4.86 | 1.68 |
| 4 | ACLT | BQ-123 | 2.83 | 1.47^{a} |
| 5 | ACLT | R-954 | 2.50 | 1.22^{b} |
| 6 | ACLT | BQ-123 + R-954 | 2.67 | 1.03^{c} |

*Radiological scoring of the DX views of the knee joints [42, 43] indicated that antagonist treatment protected joint radiomorphology after ACLT. One-way ANOVA with Holm post-hoc:
^{a}P = 0.0214, ACLT/BQ-123 treatment versus ACLT/saline treatment;
^{b}P = 0.0020, ACLT/R-954 treatment versus ACLT/saline treatment;
^{c}P = 0.0125, ACLT/BQ-123 + R-954 dual treatment versus ACLT/saline treatment.

Antagonism Protects Joint Histomorphology

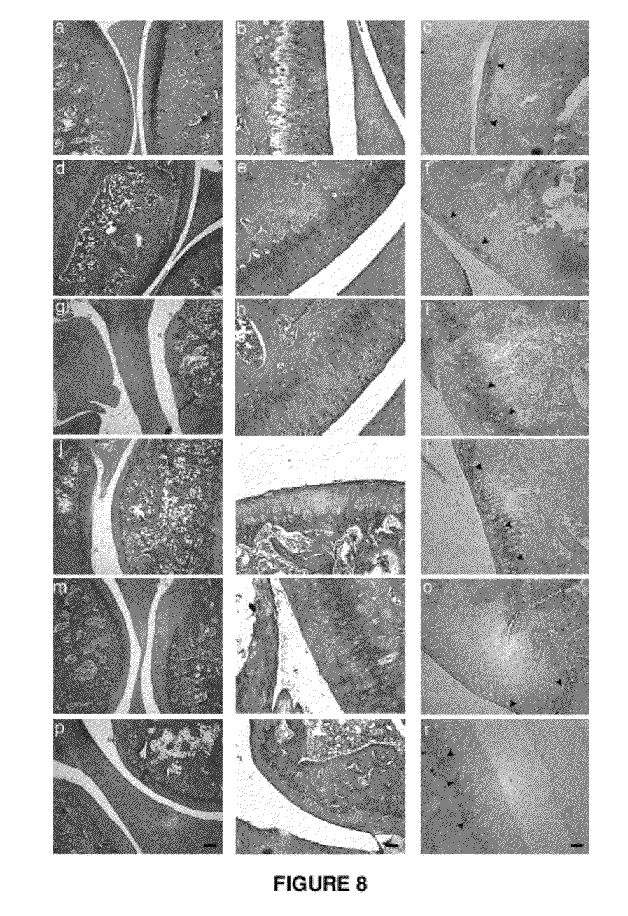

To investigate the effects of ETA and/or BKB1 antagonist treatment on histological indices of disease, rat knee joints were processed to assess cartilage proteoglycan content and joint histomorphology (FIG. 8, left and middle columns). ACLT saline-treated animals lost most proteoglycan staining when examined at eight weeks post-operatively, with severe articular surface disruptions and osteophyte formation (FIG. 8g,h). In contrast, cartilage proteoglycans were detected in the knees of ETA and/or BKB1 antagonist-treated animals (FIG. 8j,k,m,n and 8p,q), indicating that treatment protects cartilage structural components. As well, articular surface integrity was preserved to a greater extent, with dual antagonism appearing to be most protective (FIG. 8p,q). Neither sham surgery nor intraarticular injection of saline vehicle negatively affected joint histomorphology (FIG. 8a,b and 8d,e). Mean OARSI scores (Table 4) indicate that ETA and/or BKB1 antagonist treatment significantly reduced the amount of affected joint tissue and the degree of histopathology, as compared to saline-treated positive controls (P<0.0001 for all comparisons, one-way ANOVA with Holm post-hoc).

Type II collagen, the major structural collagen of cartilage, was detected by immunohistochemistry (FIG. 8, right column). ACLT saline-treated animals displayed significant losses of articular surface type II collagen (FIG. 8i) with some localization in the deep zones of cartilage, reflecting cartilage remodeling processes. Animals treated with ETA and/or BKB1 antagonists (FIG. 8l,o,r) displayed varying degrees of protection, retaining some type II collagen staining. Neither sham surgery nor intra-articular injection of saline vehicle negatively affected joint type II collagen expression (FIG. 8c,f): protein was localized in the superficial zone of articular cartilage, indicating functional joint tissue.

TABLE 4
OARSI histopathology scores

| | | | Mean OARSI | Standard |
|---|---|---|---|---|
| Group Number | Surgery | Treatment | score* | deviation |
| 1 | None | Saline | 0.43 | 0.53 |
| 2 | Sham | Saline | 0.00 | 1.00 |
| 3 | ACLT | Saline | 17.00 | 5.77 |
| 4 | ACLT | BQ-123 | 10.25 | 0.96^{a} |
| 5 | ACLT | R-954 | 4.25 | 2.02^{b} |
| 6 | ACLT | BQ-123 + R-954 | 3.50 | 2.89^{c} |

*Four slides per condition were scored by two blinded examiners using the OARSI histopathology assessment system [Pritzker K P H, Gay S, Jimenez S A, Ostergaard K, Pelletier J P, Revell P A, Salter D, van den Berg W B: Osteoarthritis cartilage histopathology: grading and staging. Osteoarthr Cartil 2006, 14: 13-29.]. Results were averaged and are presented as mean scores per condition. Inter-examiner variation was within ±5%. One-way ANOVA with Holm post-hoc:
^{a}P = 0.000017, ACLT/BQ-123 treatment versus ACLT/saline treatment;
^{b}P = 0.00001, ACLT/R-954 treatment versus ACLT/saline treatment;
^{c}P = 0.0000048, ACLT/BQ-123 + R-954 dual treatment versus ACLT/saline treatment.

Discussion

The present example investigated whether antagonism of ETA and/or BKB1 could slow and/or prevent osteoarthritic cartilage degradation and joint pain in a rat surgical model of OA. As provided herein, several lines of evidence suggest protective effects of ETA and BKB1 dual antagonism in vivo: antagonist treatment improved hind limb pain tolerance, ameliorated radiological indices of disease, and protected cartilage biochemical integrity and articular cartilage and bone histomorphometry.

The most interesting finding of our study is that pain tolerance was augmented in the animal model after ETA and/or BKB1 antagonist treatment. These results are consistent with other reports [De-Melo J D, Tonussi C R, D'Orléans-Juste P, Rae G A: Articular nociception induced by endothelin-1, carrageenan and LPS in naive and previously inflamed knee-joints in the rat: inhibition by endothelin receptor antagonists. Pain 1998, 77:261-9; Tonussi C R, Ferreira S H: Bradykinin-induced knee joint incapacitation involves bradykinin B2 receptor mediated hyperalgesia and bradykinin B1 receptor-mediated nociception. Eur J Pharmacol 1997, 326:61-5.] where local treatment with ETA or BKB1 receptor antagonists reduces overt acute joint pain. The present example extends this finding to the dual antagonist paradigm in a model of chronic pain, as well as relating it to measures of joint integrity by radiology and histology. Low-grade joint pain is the most common reason for patient presentation, and is often the major debilitating factor in OA cases. Thus, the synergistic effects of dual inhibition of ETA and BKB1 signaling make this treatment strategy more attractive than single receptor antagonism, due to the potential for lower overall drug doses.

It was found in this example that single and dual ETA/BKB1 antagonist treatments decreased radiological disease indices, in terms of osteophyte formation, cartilage thinning, and subchondral bone remodeling, with dual antagonism being most protective. As well, cartilage T2, increased in ACLT animals, was decreased by antagonist treatment, which indicates a cartilage-preserving effect. Longer cartilage transverse relaxation times are an indicator of cartilage degradation; this MR parameter is indicative of cartilage composition and integrity [Blumenkrantz G, Majumdar S: Quantitative magnetic resonance imaging of articular cartilage in osteoarthritis. European cells & materials 2007, 13:76-86; Bolbos R I, Zuo J, Banerjee S, Link T M, Ma C B, Li X, Majumdar S: Relationship between trabecular bone structure and articular cartilage morphology and relaxation times in early OA of the knee joint using parallel MRI at 3 T. Osteoarthr Cartil 2008, 16(10):1150-9; Stahl R, Luke A, Li X, Carballido-Gamio J, Ma C B, Majumdar S, Link T M: T1 rho, T2 and focal knee cartilage abnormalities in physically active and sedentary healthy subjects versus early OA patients—a 3.0-Tesla MRI study. Eur Radiol 2009, 19:132-43.]. Radiographic evidence is the main criterion for OA diagnosis and progression [Abadie E, Ethgen D, Avouac B, Bouvenot G, Branco J, Bruyere O, Calvo G, Devogelaer J P, Dreiser R L, Herrero-Beaumont G, Kahan A, Kreutz G, Laslop A, Lemmel E M, Nuki G, Van De Putte L, Vanhaelst L, Reginster J Y: Recommendations for the use of new methods to assess the efficacy of disease-modifying drugs in the treatment of osteoarthritis. Osteoarthr Cartil 2004, 12:263-268; Ornetti P, Brandt K, Hellio-Le Graverand M P, Hochberg M, Hunter D J, Kloppenburg M, Lane N, Maillefert J F, Mazzuca S A, Spector T, Utard-Wlerick G, Vignon E, Dougados M: OARSI-OMERACT definition of relevant radiological progression in hip/knee osteoarthritis. Osteoarthr Cartil 2009, 17:856-863.]. We were able to detect radiological evidence of OA progression in ACLT animals, similar to other studies [Chou M C, Tsai P H, Huang G S, Lee H S, Lee C H, Lin M H, Lin C Y, Chung H W: Correlation between the MR T2 value at 4.7 T and relative water content in articular cartilage in experimental osteoarthritis induced by ACL transection. Osteoarthr Cartil 2009, 17:441-7; Wang Y X: In vivo magnetic resonance imaging of animal models of knee osteoarthritis. Lab Anim 2008, 42:246-64.]

As previously described [Appleton C T G, McErlain D D, Pitelka V, Schwartz N, Bernier S M, Henry J L, Holdsworth D W, Beier F: Forced mobilization accelerates pathogenesis: characterization of a preclinical surgical model of osteoarthritis. Arthritis Res Ther 2007, 9:R13; Hayami T, Pickarski M, Zhuo Y, Wesolowski G A, Rodan G A, Duong L T: Characterization of articular cartilage and subchondral bone changes in the rat anterior cruciate ligament transection and meniscectomized models of osteoarthritis. Bone 2006, 38(2):234-43.], we observed that OA induction in rat knees led to a rapid decrease in cartilage proteoglycan staining, along with articular surface disruption and osteophyte formation. ETA/BKB1 antagonist treatment protected the proteoglycan content of the joint and preserved articular surface integrity. This allowed the joint cartilage to retain its normal biophysical properties, as cartilage proteoglycans are responsible, along with collagen, for retaining water in the tissue, which provides spring and resilience [Muir H: Proteoglycans of cartilage. J Clin Pathol Suppl (R Coll Pathol) 1978, 12:67-81; Roughley P J: The structure and function of cartilage proteoglycans. European cells & materials 2006, 12:92-101.]. These findings suggest that the protection of cartilage proteoglycans and articular surface histomorphology may be one explanation for the increased pain tolerance observed in antagonist-treated animals; the results presented herein concur with those of other reports, which correlated the preservation of articular cartilage proteoglycan staining with pain tolerance behaviour [Appleton C T G, McErlain D D, Pitelka V, Schwartz N, Bernier S M, Henry J L, Holdsworth D W, Beier F: Forced mobilization accelerates pathogenesis: characterization of a preclinical surgical model of osteoarthritis. Arthritis Res Ther 2007, 9:R13.].

The ET-1 and BK systems are involved in joint tissue inflammation and nociception, concomitant with pro-inflammatory mediators. However, exploration of potential therapeutic targets in these systems has been modest: the main classes of disease-modifying osteoarthritis drugs currently in development include cytokine and matrix metalloproteinase inhibitors, anti-resorptives, and growth factors [Qvist P, Bay-Jensen A C, Christiansen C, Dam E B, Pastoureau P, Karsdal M A: The disease modifying osteoarthritis drug (DMOAD): Is it in the horizon? Pharmacol Res 2008, 58:1-7.]. To the inventors' knowledge, the only clinical trial of a drug targeting a vasoactive factor in OA is the bradykinin receptor B2 antagonist Icatibant, by Sanofi-Aventis [Sanofi-Aventis. Efficacy and Safety Study of Intra-Articular Multiple Doses of Icatibant in Patients With Painful Knee Osteoarthritis. 2006-2007. Clinicaltrials.gov Identifier: NCT00303056. [http://clinicaltrials.gov/show/NCT00303056].]. This drug is no longer in clinical development [Read S J, Dray A: Osteoarthritic pain: a review of current, theoretical and emerging therapeutics. Expert Opin Investig Drugs 2008, 17(5):619-40.], due to mixed results: while it provided local analgesia in knee OA, no anti-inflammatory effect could be detected [Song I H, Althoff C E, Hermann K G, Scheel A K, Knetsch T, Burmester G R, Backhaus M: Contrast-enhanced ultrasound in monitoring the efficacy of a bradykinin receptor 2 antagonist in painful knee osteoarthritis compared with MRI. Annals of the Rheumatic Diseases 2009, 68:75-83.]. The present results suggest that ETA and BKB1 represent novel therapeutic targets in OA. Additional receptor antagonists could be tested in clinical trials for OA pain and tissue damage.

### Example 2

### Dual Antagonism Action is Performed Through the Control of MMP Systems

Methods: Human chondrocytes were isolated from human knee joints obtained at knee replacement surgery. Cells were cultured with 1) without treatment, 2) ET-1 10 nM: 3) ET-1 10 nM+BQ123 10 nM+dKD 100 nM, 4) ET-1 10 nM+R954 10 nM+dKD 100 nM: 5) ET-1 10 nM+R954 10 nM+dKD 100 nM 6) ET-1 10 nM+R954 10 nM+BQ123 10 nM+dKD 100 nM. Following 24 h of incubation the conditioned media were used for MMP-1 immunodetection by Western Blot. For Western Blot, 10 μl of media was electrophoresed on a 10% sodium dodecyl sulfate (SDS) discontinuous gradient polyacrylamide gel and transferred electrophoretically onto a nitrocellulose membrane (Hybond C™ extra; Amersham, Pharmacia Biotech Buckinghamshire, UK). The membranes were immersed overnight in Super Block™ Blocking buffer (Pierce, Rockford, Ill.). They were rinsed and incubated for 24 hours at 4° C. with a primary antibody specifically recognizing MMP-1 (purified mouse monoclonal antibody recognizing the 55 kDa latent form and 43 kDa active form of MMP1, Calbiochem, Canada). Following incubation with primary antibody membranes were carefully washed and reincubated for 1 hour at 4° C. with a secondary antibody, anti-mouse IgG horseradish peroxidase (POD) conjugate, (dilution 1/40000). After careful washing, detection was performed using Super Signal Ultra™.

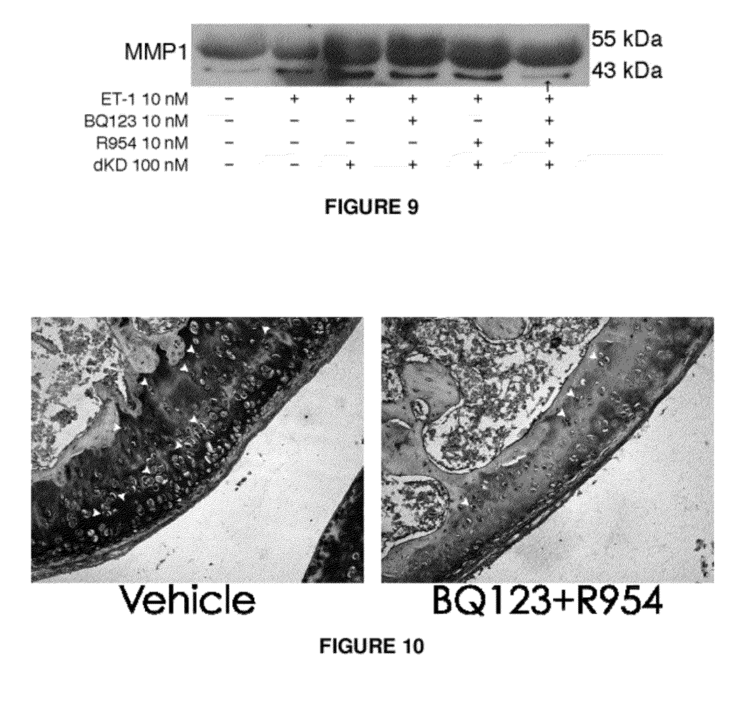

The results shown in FIG. 9 demonstrate that active 43-kDa form of MMP-1 is drastically diminished by BQ123 and R954 double inhibition (see right column, most lanes, and arrow in lower right of blot).

These results are of significance because the MMP-1 is recognized as a crucial destructive factor of cartilage matrix in OA. Therefore, inhibition of MMP-1 supports the role of a dual antagonism of ETA and BKB1 receptors in the control of the prevention and treatment of osteoarthritic cartilage degradation.

### Example 3

### Dual Antagonism Reduces BKB1 Expression

Methods: Immunohistochemistry was performed as described in Kaufman et al (Kaufman et al., Arthritis Research and Therapy, 2011, 13:R76). Briefly, rat knees were decalcified, embedded in paraffin, and 5-μm midsaggital sections were obtained. The sections were mounted on microscope slides and stained according to our laboratory's standard immunohistological procedures for BKB1 expression. Stained sections were examined by light microscopy and representative photomicrographs were taken. As seen in FIG. 10, dual antagonism of ETA-R and BKB1 decreases BKB1 expression in OA rat knee joint. This effect could be an explanation of the improvement in nociceptive tolerance that was seen upon bradykinin receptor antagonism (published in Kaufman et al., Arthritis Research and Therapy, 2011, 13:R76), because BKB1 is a receptor implicated in articular nociception and pro-inflammatory reactions.

### Example 4

### Detection of COX-2 by Immunohistochemistry

Method: 5-micron sections were processed for immunohistochemical detection of cyclooxygenase COX-2 as described above.

Results: COX-2 is an inducible enzyme undetectable in most normal tissues, but abundant in cells at sites of inflammation. This is an enzyme responsible for the formation of important biological mediators involved in pain and inflammation, such as prostaglandins, prostacyclin and thromboxane. It is well known that non-steroidal anti-inflammatory drugs, such as aspirin and ibuprofen, exert their effects through the inhibition of COX-2. FIG. 11 demonstrates specific staining for COX-2 on non-treated tissues (FIG. 11A). This staining is diminished in tissues exposed to BQ123 (FIG. 11B) or

R954 (FIG. 11C), and inhibited by the BQ123+R954 dual treatment (FIG. 11D). This is confirmed by cell count percentages (FIG. 11E).

### GENERAL CONCLUSIONS

Using a rat surgically induced model of OA, the present examples demonstrate that local treatment with specific peptide antagonists of ETA and/or BKB1 may slow or stabilize the development of radiomorphological and histomorphological changes occurring in OA pathogenesis. Furthermore, the examples showed that antagonist treatment accelerated recovery of, and improved longitudinally, pain tolerance in ACLT animals. The results presented herein also demonstrate that dual antagonism protects the joint morphology (anatomical structure), these effects occurring primarily through collagen type II and proteoglycans protection. Dual antagonism action is also performed through the control of MMP systems.

Taken together, these results indicate that blocking ETA and BKB1 improves OA prognostic indices, which implies that defective signaling might play a role in chronic OA pain. These results also support the concept of targeting dual receptor antagonism as a relevant therapeutic option.

Headings are included herein for reference and to aid in locating certain sections. These headings are not intended to limit the scope of the concepts described therein under, and these concepts may be applicable in other sections throughout the entire specification. Thus, the present invention is not intended to be limited to the embodiments shown herein but is to be accorded the widest scope consistent with the principles and novel features disclosed herein.

As used herein and in the appended claims, the singular forms “a”, “an”, and “the” include plural referents unless the context clearly indicates otherwise. Thus, for example, reference to “a compound” includes one or more of such compounds, and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and so forth.

It is understood that the examples and embodiments described herein are for illustrative purposes only and that various modifications or changes in light thereof will be suggested to persons skilled in the art and are to be included within the present invention and scope of the appended claims.

1. A method for treating pain in a subject in need thereof, comprising administering to said subject a combination of (i) an antagonist of the endothelin type A receptor (ETA) and (ii) an antagonist of the bradykinin B1 receptor (BKB1).

2. The method of claim 1, wherein said pain is selected from the group consisting of: pain initiated by inflammation; pain caused by as stimulation of nociceptors in the peripheral nervous system; pain caused by damage to, or malfunction of, the peripheral or central nervous system; neuropathic pain; vascular pain; deep somatic pain; diffuse visceral pain and chronic pain.

3. The method of claim 1, wherein said pain is associated with a disease or condition selected from the group consisting of osteoarthritis, ankylosing spondylitis, joint manifestation of Behçet's disease, fibromyalgia, Ehlers-Danlos syndrome, Gout, infectious arthritis, Felty's syndrome, juvenile arthritis, systemic lupus erythematosus, mixed connective tissue disease (MCTD), repetitive stress injury, chronic back injury, and carpal tunnel syndrome.

4. The method of claim 3, wherein said pain is joint pain and wherein said disease is osteoarthritis.

5. The method of claim 1, wherein said antagonist of endothelin type A receptor (ETA) is selected from the group consisting of BQ-123, Bosentan (Ro-470203), Atransentan (ABT627), Tezosentan (Ro-610612), Sitaxsentan (TBC-11251), Darusentan (LU-135252), Clazosentan (Ro61-1790, AXV-034343), ZD-4054, Ambrisentan (LU-208075), TAK-044, and Avosentan (SPP301).

6. The method of claim 1, wherein said antagonist of a bradykinin b1 receptor (BKB1) is selected from the group consisting of R-954, MK-0686, SSR-240612, AMG-379, comprising Formula (I) as defined in WO 2006/017938 and compounds of Formula (I) as defined in U.S. Pat. No. 7,211,566.

7. The method of claim 1, wherein said antagonist of the endothelin type A receptor (ETA) consists of compound BQ-123, or a pharmaceutically acceptable salt thereof; and wherein said antagonist of the bradykinin B1 receptor (BKB1) consists of compound R-954, or a pharmaceutically acceptable salt thereof:

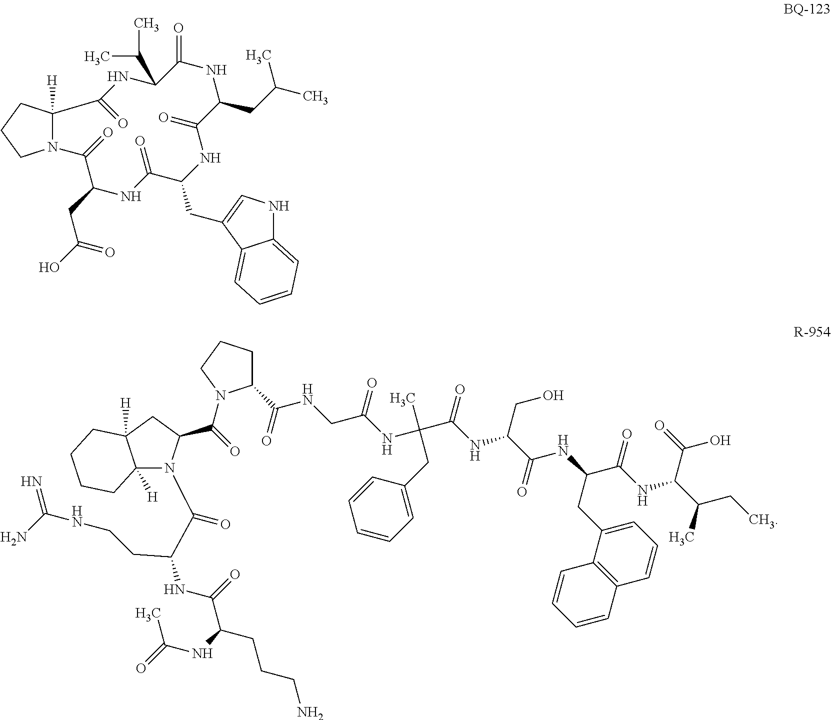

8. The method of claim 1, wherein said administration comprises administering said combination locally at a site where the subject feels pain.

9. The method of claim 8, wherein said administration comprises administering said combination by injection.

10. A method for treating pain in a subject suffering from osteoarthritis, comprising administering to said subject a combination of (i) an antagonist of the endothelin type A receptor (ETA) and (ii) an antagonist of the bradykinin B1 receptor (BKB1).

11. The method of claim 10, wherein said antagonist of the endothelin type A receptor (ETA) consists of compound BQ-123, or a pharmaceutically acceptable salt thereof; and wherein said antagonist of the bradykinin B1 receptor (BKB1) consists of compound R-954, or a pharmaceutically acceptable salt thereof:

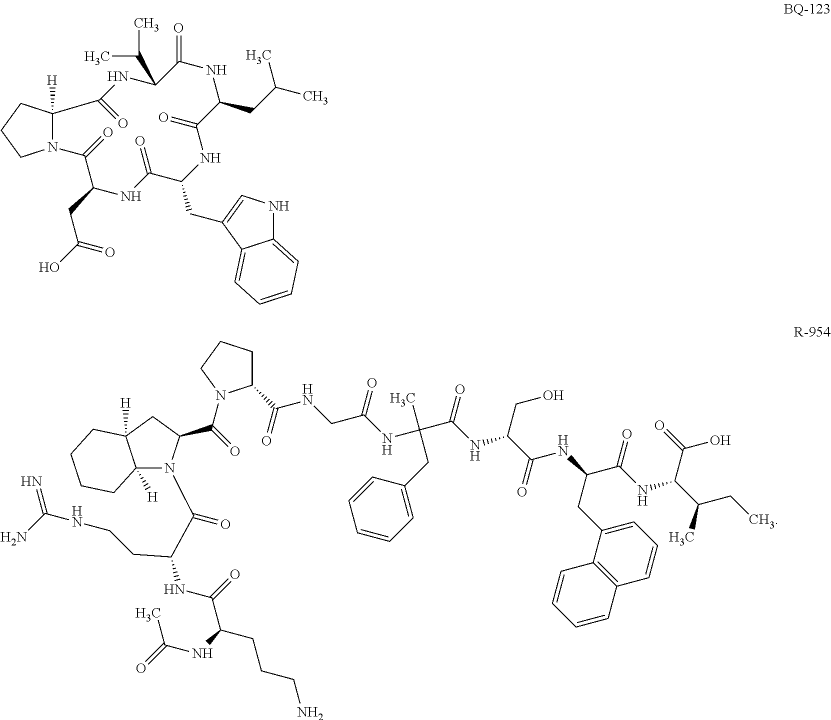

12. The method of claim 1, wherein said administration comprises administering said combination by injection locally at a site where the subject feels pain.

13. A method for preventing osteoarthritic cartilage degradation in a subject in need thereof, comprising administering to said subject a combination of (i) an antagonist of the endothelin type A receptor (ETA) and (ii) an antagonist of the bradykinin B1 receptor (BKB1).

14. The method of claim 13, wherein said cartilage degradation is a degradation of cartilage of the elbow, cartilage of the knee, cartilage of the ankle or foot, cartilage of the hand joints, cartilage of the hip and/or cartilage of the intervertebral discs, and/or cartilage of the growth plate.

15. The method of claim 13, wherein said antagonist of the endothelin type A receptor (ETA) consists of compound BQ-123, or a pharmaceutically acceptable salt thereof; and wherein said antagonist of the bradykinin B1 receptor (BKB1) consists of compound R-954, or a pharmaceutically acceptable salt thereof:

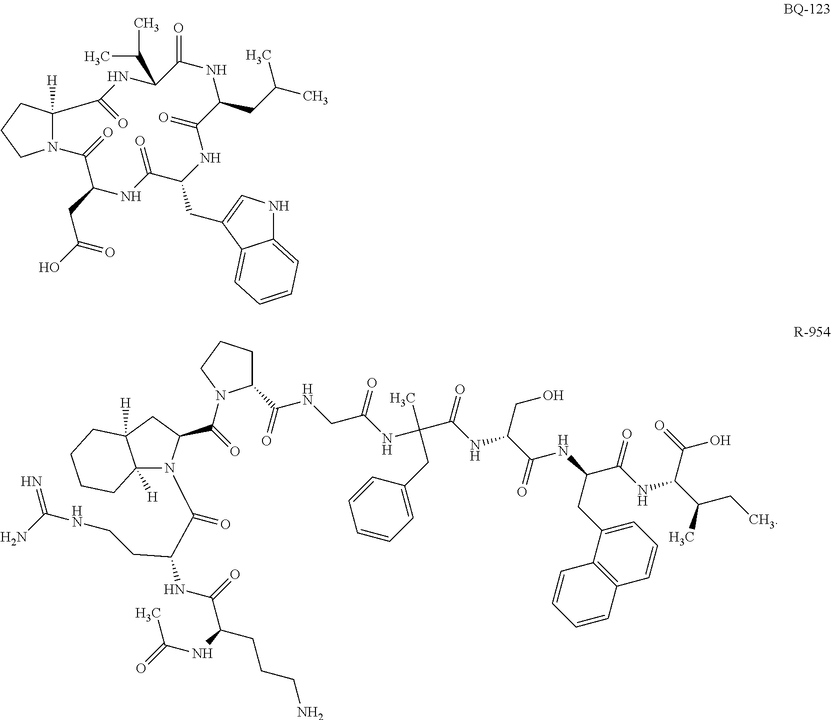

16. The method of claim 13, wherein said administration comprises administering said combination by injection locally at a site of cartilage degradation.

17. A pharmaceutical composition comprising pharmaceutically acceptable vehicle and a combination of (i) an antagonist of the endothelin type A receptor (ETA) and (ii) an antagonist of the bradykinin B1 receptor (BKB1) for treating pain, for preventing osteoarthritic cartilage degradation, and/or for preventing osteoarthritic joint inflammation, in a subject in need thereof.

18. The pharmaceutical composition of claim 17, wherein said antagonist of endothelin type A receptor (ETA) is selected from the group consisting of BQ-123, Bosentan (Ro-470203), Atransentan (ABT627), Tezosentan (Ro-610612), Sitaxsentan (TBC-11251), Darusentan (LU-135252), Clazosentan (Ro61-1790, AXV-034343), ZD-4054, Ambrisentan (LU-208075), TAK-044, and Avosentan (SPP301).

19. The pharmaceutical composition of claim 17, wherein said antagonist of a bradykinin b1 receptor (BKB1) is selected from the group consisting of R-954, MK-0686, SSR-240612, AMG-379, comprising Formula (I) as defined in WO 2006/017938 and compounds of Formula (I) as defined in U.S. Pat. No. 7,211,566.

20. The pharmaceutical composition of claim 17, wherein said antagonist of the endothelin type A receptor (ETA) consists of compound BQ-123, or a pharmaceutically acceptable salt thereof; and wherein said antagonist of the bradykinin B1 receptor (BKB1) consists of compound R-954, or a pharmaceutically acceptable salt thereof:

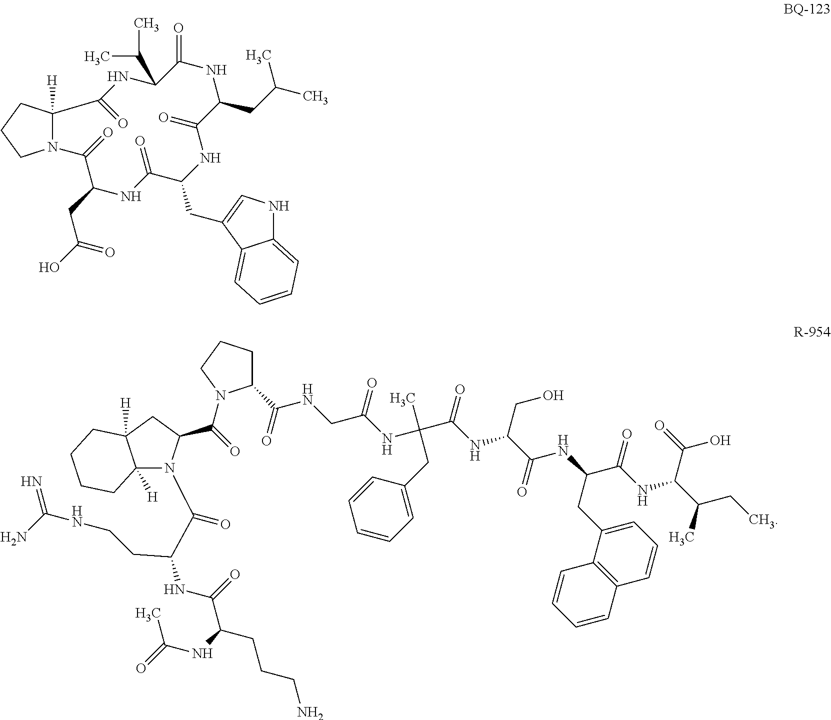

21. The pharmaceutical composition of claim 17, wherein said composition is formulated for a local injection in a human subject at a site of pain, at a site of osteoarthritic cartilage degradation, and/or at a site of joint inflammation.
